(19) United States
(12) Patent Application Publication
(10) Pub. No.: US 2024/0382416 A1
(43) Pub. Date: Nov. 21, 2024

(54) METHOD OF TREATING INFLAMMATION AND PHOTORECEPTOR CELL DEGENERATION IN RETINAL DEGENERATION PATIENTS

(71) Applicants: Centre for Eye and Vision Research Limited, Hong Kong (HK); The Hong Kong Polytechnic University, Hong Kong (HK)

(72) Inventors: Rong LI, Hong Kong (HK); Bin LIN, Hong Kong (HK)

(21) Appl. No.: 18/592,423

(22) Filed: Feb. 29, 2024

Related U.S. Application Data
(60) Provisional application No. 63/503,491, filed on May 21, 2023.

(51) Int. Cl. A61K 9/00

(52) U.S. Cl. CPC A61K 9/0048

(57) ABSTRACT

A pharmaceutical composition for treating retinal degeneration is provided. Particularly, the pharmaceutical composition includes a tropomyosin 1 (TPM1) inhibitor and is further enriched with pharmaceutically acceptable additives. In addition, the pharmaceutical formulation is configured to suppress and/or knock down the expression and/or activity of TPM1, thereby addressing the molecular factors associated with retinal degeneration.

### CROSS-REFERENCE WITH RELATED APPLICATIONS

The present application claims priority from the U.S. Provisional Patent Application No. 63/503,491 filed May 21, 2023, and the disclosure of which is incorporated herein by reference in its entirety.

### REFERENCE TO SEQUENCE DISCLOSURE

The sequence listing file under the file name “P2761US01_Sequence Listing.xml” submitted in ST.26 XML file format with a file size of 21.6 KB created on Feb. 2, 2024 and filed on Feb. 29, 2024 is incorporated herein by reference.

### FIELD OF THE INVENTION

The present invention generally relates to ophthalmology fields. More specifically the present invention relates to a method of treating retinal degeneration.

### BACKGROUND OF THE INVENTION

Retinal degeneration (RD) stands as a prominent cause of irreversible blindness globally, attributed to factors such as gene mutations, aging, environmental changes, or cellular damage within the retina (Chang et al., 2002; Majidi et al., 2021; Wert et al., 2014). Age-related macular degeneration (AMD) and retinitis pigmentosa (RP) represent two prevalent forms of RD. AMD involves the degeneration of photoreceptor cells, particularly in the central macular region, leading to central vision loss. RP, on the other hand, comprises a cluster of eye diseases marked by progressive photoreceptor degeneration, eventually resulting in partial or complete blindness. The substantial impact of vision loss on quality of life is evident through diminished social interaction, reduced independence, and impaired mobility. Global estimates from 2015 indicate that approximately 253 million people suffered from visual impairment, with the prevalence of blindness and moderate to severe visual impairment (MSVI) continually rising (Ackland et al., 2017; Burton et al., 2021).

The mechanisms underpinning photoreceptor degeneration in RD remain incompletely elucidated. Neuroinflammation has been implicated in the pathological progression of RD; however, the molecular intricacies of proinflammatory responses in RD are poorly understood, and specific treatments for RD are currently lacking. Despite attempts through clinical interventions and innovative treatments to rescue or slow down retinal degeneration, a comprehensive approach to address this healthcare challenge is still elusive (Majidi et al., 2021).

Therefore, the present invention addresses this need.

### SUMMARY OF THE INVENTION:

It is an objective of the present invention to provide methods to solve the aforementioned technical problems.

In accordance with a first aspect of the present invention, a pharmaceutical composition including a tropomyosin 1 (TPM1) inhibitor for treating a retinal degeneration in a subject in need thereof is provided. Furthermore, the composition further includes a pharmaceutically acceptable addition.

In accordance with one embodiment of the present invention, the pharmaceutical composition suppresses and/or knocks down the expression and/or activity of the TPM1.

In accordance with one embodiment of the present invention, the TPM1 inhibitor includes a triggering receptor expressed on myeloid cells 2 (TREM2) silencer.

In accordance with one embodiment of the present invention, the refractive disorder is selected from age-related macular degeneration (AMD) or retinitis pigmentosa (RP).

In accordance with one embodiment of the present invention, the pharmaceutically acceptable addition includes an excipient, a stability additive, a carrier, a diluent, and a solubilizer.

In accordance with one embodiment of the present invention, the pharmaceutical composition is formulated to an administration form that enables delivery to the subject's subretinal space through the cornea and/or the blood-retinal barrier.

In accordance with one embodiment of the present invention, the administration form is selected from an immediate-release form or a controlled-release form.

In accordance with one embodiment of the present invention, the pharmaceutical composition is delivered through an approach selected from an intravitreal injection, a subretinal injection or a suprachoroidal injection.

In accordance with one embodiment of the present invention, the administration form includes an injection form, an eye drop form, an eye ointment form, a hydrogel form, an ultrasonic ocular drug delivery form, a drug-loaded contact lenses form, a drug-eluting implant form, a nanoparticle-mediated delivery, an intravitreal gene therapy form, and an intravitreal microneedle form.

In accordance with a second aspect of the present invention, a method for reducing neuroinflammation in a subject in need thereof, including administering the composition including the TPM1 inhibitor and a pharmaceutically acceptable addition to the subject.

In accordance with a third aspect of the present invention, a method for rescuing visual function in a subject in need thereof, including administering the aforementioned composition to the subject.

In accordance with one embodiment of the present invention, the photoreceptor degeneration is reversed in the subject.

In accordance with a fourth aspect of the present invention, a usage of the aforementioned pharmaceutical composition for treating a retinal degeneration in a subject in need thereof.

In accordance with one embodiment of the present invention, the retinal degeneration is selected from AMD or RP.

In accordance with one embodiment of the present invention, the pharmaceutical composition reduces neuroinflammation in the subject.

In accordance with one embodiment of the present invention, the pharmaceutical composition reverses the photoreceptor degeneration and rescues visual function in the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

Embodiments of the invention are described in more details hereinafter with reference to the drawings, in which:

FIGS. 1A-1C illustrate the upregulation of TPM1 in the rd 10 mouse model of RP, in which FIG. 1A shows the Western blot analysis of TPM1 in rd10 and C57BL/6J mice at P16, P19, P22 and P25, with the quantification displayed in FIG. 1B, and FIG. 1C demonstrates the qPCR analysis of Tpm1 in rd10 and C57BL/6J mice at P16, P19, P22 and P25;

FIGS. 2A-2N exhibits the genetic knockdown of TPM1 ameliorates inflammation and photoreceptor degeneration in rd10 mice, in which FIGS. 2A-2F respectively show the qPCR analysis of Tpm1 (FIG. 2A), TNF-a (FIG. 2B), IL-1β (FIG. 2C), IL-6 (FIG. 2D), COX-2 (FIG. 2E) and iNOS (FIG. 2F) in rd10 mice treated with siTPM1 or siCTR, and C57BL/6J mice at P25. n=4 mice in each group, FIG. 2G displays the retina sections stained with Iba-1 and CD68 antibodies and FIG. 2H shows the quantification of density of microglia in the outer nuclear layer (ONL) of rd10 retinas treated with siTPM1 or siCTR, and C57BL/6J retinas at P25 with yellow arrows indicating the resting microglia and white arrows showing the migration of activated microglia to the ONL, FIG. 2I depicts the retina sections stained with GFAP antibody in rd10 mice treated with siTPM1 or siCTR, and C57BL/6J mice at P25 with yellow arrows indicating the resting astrocytes and white arrows specifying activated astrocytes and Müller cells in the retina, FIG. 2J displays the quantification of GFAP immunofluorescence intensity in rd10 mice treated with siTPM1 or siCTR, and C57BL/6J mice at P25, FIG. 2K shows the TUNEL staining (white arrows indicating the TUNEL-positive cells in the ONL of the retina) and FIG. 2L depicts the quantification of TUNEL-positive cells in the ONL of rd10 retinas treated with siTPM1 or siCTR, and C57BL/6J retinas at P25, FIG. 2M and FIG. 2N respectively show the scotopic and photopic electroretinogram (ERG) recordings on rd10 mice treated with siTPM1 or siCTR, and C57BL/6J mice at P25;

FIGS. 3A-3N depict that TPM1 overexpression exacerbates the retinal glial reaction and photoreceptor loss in rd10 mice, in which FIGS. 3A-3F respectively display the qPCR analysis of Tpm1 (FIG. 3A), TNF-a (FIG. 3B), IL-1β (FIG. 3C), IL-6 (FIG. 3D), COX-2 (FIG. 3E) and iNOS (FIG. 3F) in rd10 mice treated with AAV-EGFP-TPM1 or AAV-EGFP at P25, FIG. 3G shows the retina sections stained with Iba-1 antibody (white arrows indicating the migration of activated microglia to the ONL) and FIG. 3H exhibits the quantification of microglia density in the ONL from rd10 retinas treated with AAV-EGFP-TPM1 or AAV-EGFP at P25, FIG. 3I depicts the retina sections stained with GFAP (white arrows indicating the activated astrocytes and Müller cells in retina) and FIG. 3J demonstrates the quantification of GFAP immunofluorescence intensity in rd10 retinas treated with AAV-EGFP-TPM1 or AAV-EGFP at P25, FIG. 3K depicts the TUNEL staining and FIG. 3L displays the TUNEL-positive cells (white arrows) in the retina from rd10 mice treated with AAV-EGFP-TPM1 or AAV-EGFP at P25, and FIG. 3M and FIG. 3N respectively shows the ERG recording on rd10 mice treated with AAV-EGFP-TPM1 or AAV-EGFP at P25;

FIGS. 4A-4X indicate that TREM2 knockout downregulates TPM1 and inhibits the retinal glial reaction and photoreceptor degeneration in rd10 mice, in which FIG. 4A depicts the qPCR analysis of TREM2 expression in rd10 and C57BL/6J mice at P16, P19, P22 and P25. n=4 and 6 mice in C57BL/6J and rd10 respectively, FIG. 4B shows the western blot analysis of TREM2 in rd10 and C57BL/6J mice at P16, P19, P22 and P25, with the quantification results presented in FIG. 4C, FIGS. 4D-4H respectively depict the qPCR analysis of TNF-a (FIG. 4D), IL-1β (FIG. 4E), IL-6 (FIG. 4F), COX-2 (FIG. 4G) and iNOS (FIG. 4H) in TREM2^{-/-}/rd10, rd10 and C57BL/6J mice at P25, FIG. 4I shows the retina whole-mounts stained with antibodies against Iba-1 and CD68 in the inner plexiform layer (IPL) and outer plexiform layer (OPL) of the retina from TREM2^{-/-}/rd10, rd10 and C57BL/6J mice at P25 (yellow arrows showing the resting microglia and white arrows indicating the activated microglia), with the quantifications of Iba-1⁺ microglia number and Iba-1⁺CD68⁺ microglia number respectively presented in FIG. 4J and FIG. 4K, FIG. 4L depicts the retina sections stained with Iba-1 and CD68 antibodies (white arrows indicating the migration of activated microglia to the ONL) and FIG. 4M displays the quantification of microglia density in the ONL of the retina from TREM2^{-/-}/rd10, rd10 and C57BL/6J mice at P25, FIG. 4N depicts the retina sections stained with GFAP antibody (yellow arrows indicating the resting astrocytes and the white arrows showing activated astrocytes and Müller cells in the retina) and FIG. 4O shows the quantification of GFAP immunofluorescence intensity in the retina of TREM2^{-/-}/rd10 and C57BL/6J mice at P25, FIG. 4P depicts the TUNEL staining and FIG. 4Q displays the quantification of TUNEL-positive cells (white arrow) in the retina of TREM2^{-/-}/rd10, rd10 and C57BL/6J mice at P25, FIG. 4R depicts the retina sections stained with R/G opsin (yellow arrows showing the healthy cone photoreceptors and the white arrows indicating the abnormal cone photoreceptors in the retina) and FIG. 4S shows the quantification of OS/IS length in the retina from TREM2^{-/-}/rd10, rd10 and C57BL/6J mice at P25, FIG. 4T and FIG. 4U shows the ERG recording on TREM2^{-/-}/rd10, rd10 and C57BL/6J mice at P25. n=12, 15 and 18 mice in C57BL/6J, rd10 and TREM2^{-/-}/rd10 respectively, FIG. 4V depicts the western blot analysis of TPM1 and p-CREB/CREB in TREM2^{-/-}/rd10 , rd10 and C57BL/6J mice at P25, and FIGS. 4W-4X depict the quantification of TPM1 (FIG. 4W) and p-CREB/CREB (FIG. 4X) in TREM2^{-/-}/rd10, rd10 and C57BL/6J mice at P25; and

FIGS. 5A-5W demonstrate that genetic overexpression of TPM1 in TREM2^{-/-}/rd10 mice increases inflammation and photoreceptor loss, in which FIGS. 5A-5H respectively depicts the qPCR analysis results of TPM1 (FIG. 5A), TNF-a (FIG. 5B), IL-1β (FIG. 5C), IL-6 (FIG. 5D), COX-2 (FIG. 5E), iNOS (FIG. 5F), IL-10 (FIG. 5G) and IL-13 (FIG. 5H) in TREM2^{-/-}/rd10 mice treated with AAV-EGFP-TPM1 and AAV-EGFP at P25, FIG. 5I shows the immunocytochemistry image of retinal whole-mounts stained with Iba-1 in the IPL and OPL of the retina from TREM2^{-/-}/rd10 mice treated with AAV-EGFP-TPM1 and AAV-EGFP at P25, FIG. 5J shows the quantification of Iba⁺ microglia (white arrows) in the IPL and OPL of the retina from TREM2^{-/-}/rd10 mice treated with AAV-EGFP-TPM1 and AAV-EGFP at P25, FIG. 5K depicts the retinal sections stained with Iba-1 (white arrow indicating migration of activated microglia to the ONL) and FIG. 5L shows the quantification of microglia density in the ONL of the retina from TREM2^{-/-}/rd10 mice treated with AAV-EGFP-TPM1 and AAV-EGFP at P25, FIG. 5M depicts the retinal sections stained with GFAP (white arrow indicating the activated astrocytes and Müller cells in retina) and FIG. 5N exhibits the quantification of GFAP immunofluorescence intensity in the retina from TREM2^{-/-}/rd10 mice treated with AAV-EGFP-TPM1 and AAV-EGFP at P25, FIG. 5O shows the TUNEL staining and FIG. 5P displays the quantification of TUNEL-positive cells (white arrow) in the retina of TREM2^{-/-}/rd10 mice treated with AAV-EGFP-TPM1 and AAV-EGFP at P25, FIG. 5Q depicts the retinal sections stained with R/G opsin (white arrow indicating the abnormal cone photoreceptors) and FIG. 5R shows the quantification of OS/IS length in the retina of TREM2^{-/-}/rd10 mice treated with AAV-EGFP-TPM1 and AAV-EGFP at P25, FIG. 5S and FIG. 5T respectively shows the a wave and b wave of ERG recording on TREM2^{-/-}/rd10 mice treated with AAV-EGFP-TPM1 and AAV-EGFP at P25, FIG. 5U shows the western blot analysis of TPM1 and p-CREB/CREB in TREM2^{-/-}/rd10 mice treated with AAV-EGFP-TPM1 and AAV-EGFP at P25, with the quantification results presented in FIG. 5V (TPM1) and FIG. 5W (p-CREB/CREB).

### DETAILED DESCRIPTION

In the following description, methods of treating retinal degeneration and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

In accordance with a first aspect of the present invention, a pharmaceutical composition designed for the treatment of retinal degeneration, a condition that significantly impacts visual function, is provided. The composition includes a tropomyosin 1 (TPM1) inhibitor, a key component in the regulation of processes associated with retinal degeneration. To ensure efficacy and safety, the pharmaceutical composition further includes various pharmaceutically acceptable additions, such as excipients, stability additives, carriers, diluents, and solubilizers.

As used herein, the term “TPM1 inhibitor” refers to a substance that interferes with or suppresses the activity or function of this specific protein, TPM1, referring to Tropomyosin alpha-1 chain, which is a protein-coding gene. In the context of drug development, a TPM1 inhibitor may be designed to modulate the biological actions of tropomyosin alpha-1 chain for therapeutic purposes. In one embodiment, the TPM1 inhibitor might be a triggering receptor expressed on myeloid cells 2 (TREM2) silencer.

One of the distinctive features of this pharmaceutical composition is its ability to suppress and/or knock down the expression and/or activity of TPM1. By targeting TPM1, the composition intervenes in the molecular processes that contribute to retinal degeneration, thereby offering a potential therapeutic solution. This inhibition of TPM1 is achieved through the incorporation of a specific TPM1 inhibitor, which, in one embodiment, includes a triggering receptor expressed on myeloid cells 2 (TREM2) silencer.

In addition to the active pharmaceutical ingredients, the pharmaceutically acceptable addition plays a crucial role in the composition. Excipients contribute to the stability and bioavailability of the formulation, while carriers aid in the delivery of the active components. Stability additives ensure the longevity of the composition, and solubilizers enhance the solubility of the active ingredients. This comprehensive formulation approach ensures that the pharmaceutical composition achieves its intended therapeutic effects with precision and efficiency.

The pharmaceutical composition is particularly designed for the treatment of refractive disorders, with a focus on age-related macular degeneration (AMD) and retinitis pigmentosa (RP). These disorders pose significant challenges to vision and are among the leading causes of visual impairment globally. By targeting TPM1, this composition aims to address the molecular pathways associated with these disorders, potentially slowing down or halting their progression.

Moreover, the composition is meticulously formulated to an administration form that enables precise delivery to the subject's subretinal space.

This strategic delivery is facilitated through the cornea and/or the blood-retinal barrier, ensuring that the pharmaceutical composition reaches the specific anatomical site where its therapeutic action is required. The administration form may take the shape of an immediate-release form or a controlled-release form, allowing flexibility in treatment modalities based on the specific requirements of the subject and the nature of the retinal degeneration being addressed.

There are several approaches for drug delivery to the subretinal space. For instance, “intravitreal injection”, a common method involves injecting drugs directly into the vitreous humor of the eye.

“Subretinal injection”, a surgical procedure involves injecting drugs directly into the subretinal space. Briefly, the vitreous gel is taken out and a bleb is raised underneath the retina, peripheral to the central retina, by injecting the pharmaceutical composition.

“Suprachoroidal approach”, the pharmaceutical composition can be given via an in-office injection that delivers the composition behind the retina into the suprachoroidal space.

Besides the common injection form, the pharmaceutical composition may be formulated in different administration forms.

“Drug-eluting implants”, such as sustained-release devices or drug-eluting micro/nanoparticles, can be surgically placed in the eye. These devices release drugs over an extended period and can be designed for targeted subretinal delivery.

“Nanoparticle-mediated delivery”, nanoparticles are engineered to encapsulate drugs and improve their bioavailability. Surface modifications can be made to enhance their affinity for the retina and facilitate subretinal delivery. In one embodiment, the composition is delivered by a nanoparticle or microemulsion drug delivery system, which enhances solubility and improve delivery efficiency by surface-conjugating active targeting agonists or improves drug solubilization capacity and bioavailability. Moreover, the systems also reduce drug toxicity, prolong the residence time, and protect biological drugs from degradation. Another direction is the integration of nanotechnology with other delivery systems, such as ultrasonic ocular drug delivery systems, drug-loaded contact lenses, and hydrogel.

“Ultrasound-mediated delivery”, uses ultrasound to enhance drug penetration into the retina. The application of ultrasound waves can transiently disrupt barriers, facilitating drug entry into the subretinal space.

“Intravitreal gene therapy”, can be delivered via intravitreal injection to target specific cells in the retina, including those in the subretinal space. Viral vectors or nanoparticles may be used to transport therapeutic genes.

“Electroporation”, involves applying electrical pulses to cells, temporarily increasing cell membrane permeability. This technique has been used for enhancing the penetration of drugs into the retina, including the subretinal space.

“Intravitreal microneedles”, are designed to penetrate the retina and deliver drugs to specific layers, including the subretinal space. This is a minimally invasive approach compared to traditional subretinal injections.

The choice of the delivery method depends on factors such as the administration form, the targeted location, the specific eye condition, and the desired duration of drug release.

In some embodiments, the administration form includes an intravitreal injection form, a subretinal injection form, an eye drop form, an eye ointment form, a hydrogel form, an ultrasonic ocular drug delivery form, a drug-loaded contact lenses form, a drug-eluting implant form, a nanoparticle-mediated delivery, an intravitreal gene therapy form, and an intravitreal microneedle form.

Drug formulation and delivery system are crucial for ophthalmic applications. The delivery of the TPM1 inhibitor is necessary to pass through the cornea and/or the blood-retinal barrier and reach the retina and/or subretinal space. The delivery technologies encompass the following characteristics: (1) easy and noninvasive administration; (2) an efficient delivery system; (3) compatible with ocular tissues; (4) target-specific for the indicated ocular diseases; and (5) a controlled-release system, which keeps active agents for a prolonged period (several months to several years) in the retina.

Sustained-release formulations of TPM1 inhibitor can be administered

once daily or even less frequently. Sustained-release formulations can be based on matrix technology. In this technology, TPM1 inhibitor is embedded in an excipient that makes a non-disintegrating core called a matrix. Diffusion of TPM1 inhibitor occurs through the core.

A pulsed-release dosage form includes an immediate-release dosage form including TPM1 inhibitor; and a delayed-release dosage form including TPM1 inhibitor.

In one embodiment, a delayed-release dosage form can be combined with an immediate-release dosage form to provide a pulsed-release dosage form. The delayed-release dosage form may be in the form of a core which optionally includes absorption enhancers and/or water swellable substances. Pulsed-release dosage forms allow for control of the plasma levels of TPM1 inhibitor.

The term of “immediate-release” means that a conventional or non-modified release form in which greater than or equal to about 75% of the active agent is released within two hours of administration, preferably within one hour of administration.

The term “controlled-release” is a dosage form in which the release of the active agent is controlled or modified over a period of time. Controlled can mean, for example, sustained, delayed or pulsed-release at a particular time. Alternatively, controlled can mean that the release of the active agent is extended for longer than it would be in an immediate-release dosage form, i.e., at least over several hours, such as greater than four hours, preferably greater than eight hours.

The term “sustained-release” or “extended-release” is meant to include the release of the active agent at such a rate that blood (e.g., plasma) levels are maintained within a therapeutic range but below toxic levels for at least about 8 hours, preferably at least about 12 hours after administration at steady-state. The term “steady-state” means that a plasma level for a given active agent has been achieved and which is maintained with subsequent doses of the drug at a level which is at or above the minimum effective therapeutic level and is below the minimum toxic plasma level for a given active agent.

The term “delayed-release” means that there is a time-delay before significant plasma levels of the active agent are achieved. A delayed-release formulation of the active agent can avoid an initial burst of the active agent, or can be formulated so that release of the active agent in eye ball or muscle layer is avoided and absorption takes places in retina.

A “pulsed-release” formulation can contain a combination of immediate-release, sustained-release, and/or delayed-release formulations in the same dosage form. A “semi-delayed-release” formulation is a pulsed-released formulation in which a moderate dosage is provided immediately after administration and a further dosage some hours after administration.

In accordance with a second aspect of the present invention, a method for reducing neuroinflammation in subjects suffering from it. Specifically, the method involves the administration of the aforementioned pharmaceutical composition to individuals in need of such intervention.

In accordance with a third aspect of the present invention, a method for rescuing visual function, where the aforementioned pharmaceutical composition is administered to a subject experiencing visual impairment. Particularly, the method not only rescues visual function but has the potential to reverse photoreceptor degeneration in the subject. This reversal marks a significant advancement in retinal degeneration treatment strategies, evidencing the therapeutic impact of the pharmaceutical composition on the underlying causes of visual impairment.

In accordance with a fourth aspect of the present invention, a usage of a pharmaceutical composition including a TPM1 inhibitor for the treatment of retinal degeneration in subjects requiring such therapeutic intervention. The usage provides a targeted approach to addressing the complexities associated with retinal degeneration, with TPM1 inhibition playing a pivotal role in mitigating the progression of the disorder. In a specific embodiment, this use is particularly effective in treating refractive disorders, wherein the retinal degeneration is identified as AMD or RP. The pharmaceutical composition, with its selected TPM1 inhibitor, demonstrates efficacy in modulating the molecular pathways involved in these specific retinal degenerative conditions.

Particularly, the pharmaceutical composition is capable of reducing neuroinflammation in subjects with retinal degeneration to stop the disease development. Furthermore, the pharmaceutical composition reverses photoreceptor degeneration and rescues visual function in the subject to cure or contain retinal degeneration.

In conclusion, the present invention provides a comprehensive method and use of a pharmaceutical composition, offering a multifaceted approach to treating retinal degeneration, rescuing visual function, and reducing neuroinflammation in subjects in need thereof. The unique combination of a TPM1 inhibitor and pharmaceutically acceptable additions positions this composition as a promising therapeutic intervention for individuals affected by retinal degeneration. The use of the terms “a” and “an” and “the” and similar referents in the

context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### EXAMPLES

### Methods

### Retina Immunoblotting

Retinas are lysed with RIPA buffer (Abcam) or T-PER™ Tissue Protein Extraction Reagent (Invitrogen) containing the cocktails of protease and phosphatase inhibitors (Roche) on ice for 30 min. After centrifugation at 15000 rpm for 15 min, the supernatants are transferred into a new and sterile Eppendorf tube. Protein quantification is performed with Pierce™ rapid gold BCA protein assay kit (Invitrogen). 20 μg of proteins are loaded into the 10% SDS-PAGE gel, and then the gel is run at 80-100 V for 90 min followed by transferred the gel to a polyvinylidene difluoride membrane at 300 mA for 120 min. After blocking with 5% BSA for 1 h, the membranes carrying proteins are incubated with primary antibodies including rabbit anti-TPM1 (ABclonal, 1:1000), mouse anti-TREM2 (Santa Cruz, 1:500), rabbit anti-Phospho-CREB (Ser133) (Cell Signaling Technology, 1:1000), rabbit anti-CREB (48H2) (Cell Signaling Technology, 1:1000), mouse anti-actin (ABclonal, 1:2000) and mouse anti-GAPDH (ABclonal, 1:2000) overnight at 4° C. After washing three times with PBST, the membranes are incubated with secondary antibodies including goat anti-rabbit IgG and goat anti-mouse IgG (Invitrogen, 1:1000) conjugated to horseradish peroxidase for 2 h at room temperature. After incubation with SuperSignal™ West Pico PLUS Chemiluminescent Substrate (Invitrogen) or ECL™ Select Western Blotting Detection Reagent (Amersham™), the membrane with protein is evaluated by ChemiDoc Imaging Systems (Bio-rad, California, USA).

### Immunocytochemistry and Confocal Imaging

After enucleation, the retina is separated from the vitreous and sclera in PBS and fixed at 4% paraformaldehyde (PFA) for 1 h followed by dehydrating with 30% sucrose overnight at 4° C. Some of retinas are serially sectioned at the thickness of 14 um using a cryostat microtome. After washing three times with PBS, the sections are incubated with Iba-1 (Wako, 1:500), CD68 (Bio-rad, 1:500) and GFAP (Dako, 1:500) in blocking buffer containing 3% normal donkey serum (NDS), 1% bovine serum albumin (BSA) and 0.3% Triton X-100 in PBS, pH 7.4, overnight at 4° C. Secondary antibodies including donkey anti-rabbit Alexa Fluor™ 488 (Invitrogen, 1:500), donkey anti-rat Alexa Fluor™ 568 (Invitrogen, 1:500) and donkey anti-rabbit Alexa Fluor™ 594 (Invitrogen, 1:500) are applied to the sections for 2 h before mounting with Dako fluorescence mounting medium. For whole-mounted retinas, two primary antibodies, Iba-1 (Wako, 1:500) and CD68 (Bio-rad, 1:500) in the blocking buffer, are applied for 1 day, followed by secondary donkey anti-rabbit Alexa Fluor™ 488 (Invitrogen, 1:500) and donkey anti-rat Alexa Fluor™ 568 (Invitrogen, 1:500) antibodies for 2-h incubation. Images are captured by a Zeiss LSM 800 Upright Confocal Microscope (Zeiss, USA) with a pixel resolution of 1,024×1,024, and Plan-Apochromat 40×/1.3 oil-immersion or 20×/0.8 objectives. Z-stack images with an interval of 0.5 μm is acquired. For the quantification of fluorescence intensity of GFAP, three areas at 100 μm (central), 1 mm (middle) and 1.8 mm (peripheral) from the optical nerve head in each retinal section are captured and analyzed with Image J software. For quantification of microglial cells, four sampling areas with 638.9 μm×638.9 μm squares along the dorsal-ventral axis of retinal whole-mounts at 200 μm and 1 mm from the optic nerve head on both sides are photographed, and the numbers of Iba-1+ and of CD68+Iba-1+ microglial cells are manually counted.

### Quantitative Real-Time PCR

In brief, total RNA is extracted with TransZol Up Plus RNA Kit (TransGen, China), and reverse transcription is performed with the TransScript® First-Strand cDNA Synthesis SuperMix (TransGen, China) or PrimeScript 1st strand cDNA Synthesis Kit (TaKaRa) before performing the real-time PCR with PerfectStart™ Green qPCR SuperMix (TransGen, China) or TB Green® Premix Ex Taq™ II (Tli RNase H Plus) (TakaRa) by QuantStudio™ 7 Flex Real-Time PCR System (Applied Biosystems™). Gene specific primers are listed as follows:

Tpm1 (Forward):
(SEQ ID NO: 01)
CTGAAGATGCTGACCGGAAGT;
Tpm1 (Reverse):
(SEQ ID NO: 02)
TGGTTACTGATCTCTCTGCAAACTC;
TNF-α (Forward):
(SEQ ID NO: 03)
ATGGCCTCCCTCTCATCAGT;
TNF-α (Reverse):
(SEQ ID NO: 04)
TTTGCTACGACGTGGGCTAC;
IL-1ß (Forward):
(SEQ ID NO: 05)
AATGCCACCTTTTGACAGTGATG;
IL-1ß (Reverse):
(SEQ ID NO: 06)
GGAAGGTCCACGGGAAAGAC;
IL-6 (Forward):
(SEQ ID NO: 07)
GGGACTGATGCTGGTGACAA;
IL-6 (Reverse):
(SEQ ID NO: 08)
AGCATTGGAAATTGGGGTAGGA;
COX-2 (Forward):
(SEQ ID NO: 09)
CTTCGGGAGCACAACAGAGT;
COX-2 (Reverse):
(SEQ ID NO: 10)
AAGTGGTAACCGCTCAGGTG;
Trem2 (Forward):
(SEQ ID NO: 11)
CTTGCTGGAACCGTCACCAT;
Trem2 (Reverse):
(SEQ ID NO: 12)
CTTGATTCCTGGAGGTGCTGT;
IL-10 (Forward):
(SEQ ID NO: 13)
GGTGAGAAGCTGAAGACCCT;
IL-10 (Reverse):
(SEQ ID NO: 14)
TCATTCATGGCCTTGTAGACACC;
IL-13 (Forward):
(SEQ ID NO: 15)
CCTGGATTCCCTGACCAACA;
and
IL-13 (Reverse):
(SEQ ID NO: 16)
ATTTTGGTATCGGGGAGGCT.

### Example 1. TPM1 is Upregulated in the rd10 Mouse Model of RP

The expression of TPM1 is investigated in the rd10 mouse model of retinitis pigmentosa (RP). The rd10 mice, obtained from Jackson Laboratory (Stock no: 004297), represent a well-established experimental model for human RP. These mice carry a mutation in the beta subunit of the rod-specific phosphodiesterase gene (PDE6β), resulting in the death of rods from postnatal day 18 (P18) and complete rod loss by P30. Notably, cone photoreceptors in rd10 mice remain essentially normal in cell density until P25 and decline to undetectable levels by 2-4 months of age. Given that mutations in the same gene have been identified in patients with autosomal recessive RP, the rd10 mouse serves as a relevant model for studying human RP.

To investigate the role of TPM1 in TREM2-mediated inflammation in RP, the TREM2^{-/-} mice are crossed with rd10 mice to generate TREM2^{-/-}/rd10 mice. All mice are housed in a 12-hour light/dark cycle with water and food ad libitum, and maintained in the Centralised Animal Facility, The Hong Kong Polytechnic University. All experimental procedures are approved by the Animal Subjects Ethics Sub-committee (ASESC) of The Hong Kong Polytechnic University and conducted in accordance with ARVO statement for the use of animals.

For the temporal expression analysis of TPM1 during photoreceptor degeneration in rd10 mice, retinas were collected at P16 (before degeneration), P19 (early degeneration), P22, and P25 (middle degeneration).

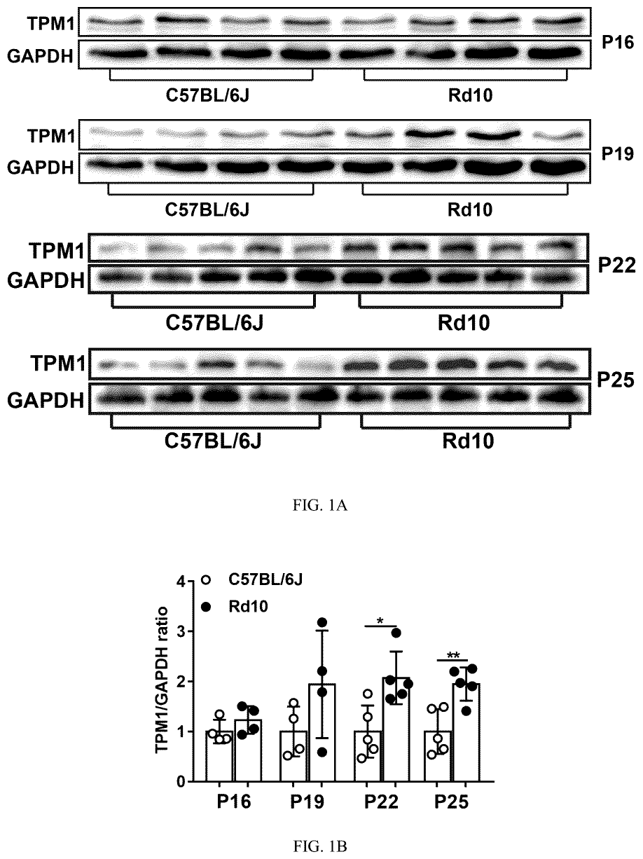

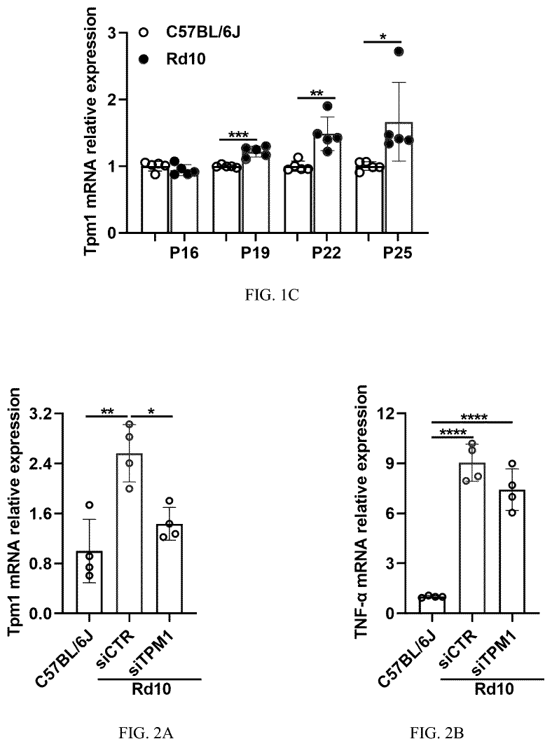

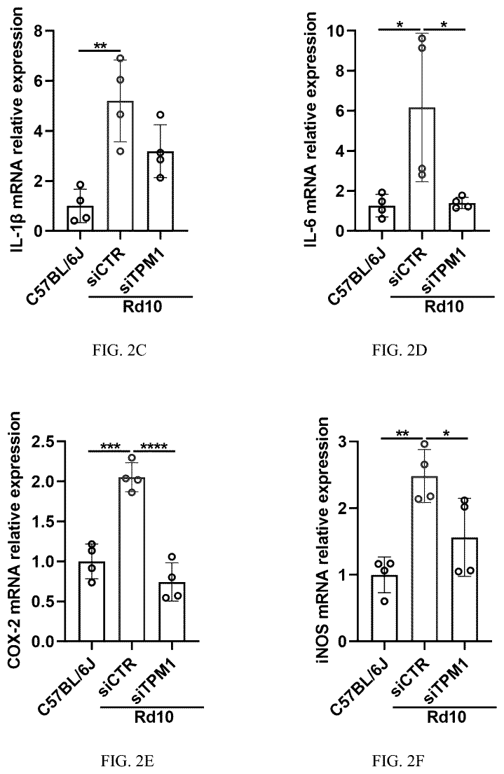

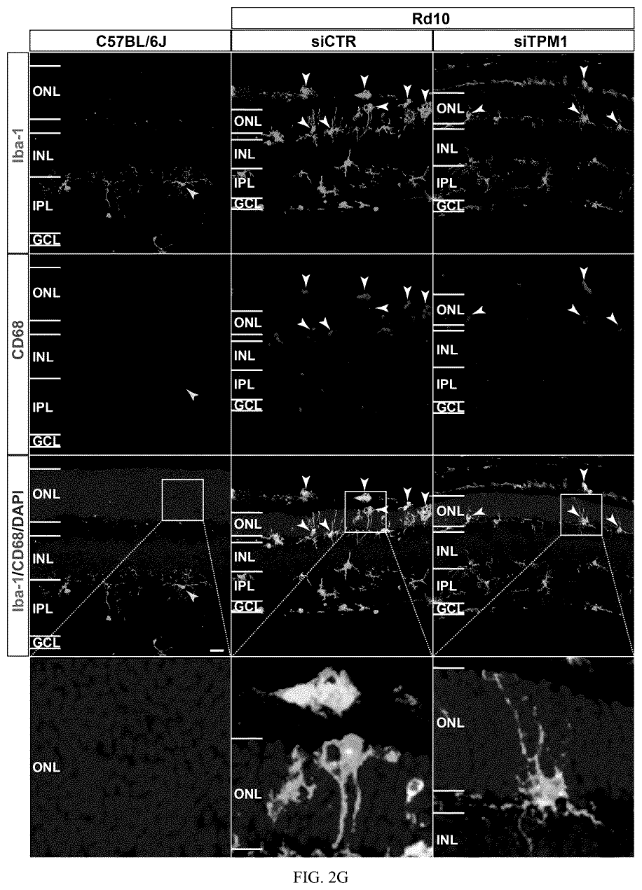

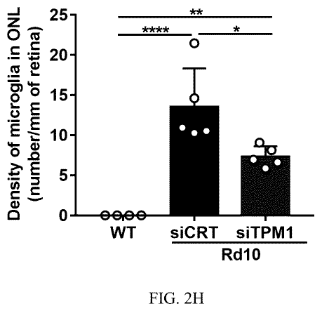

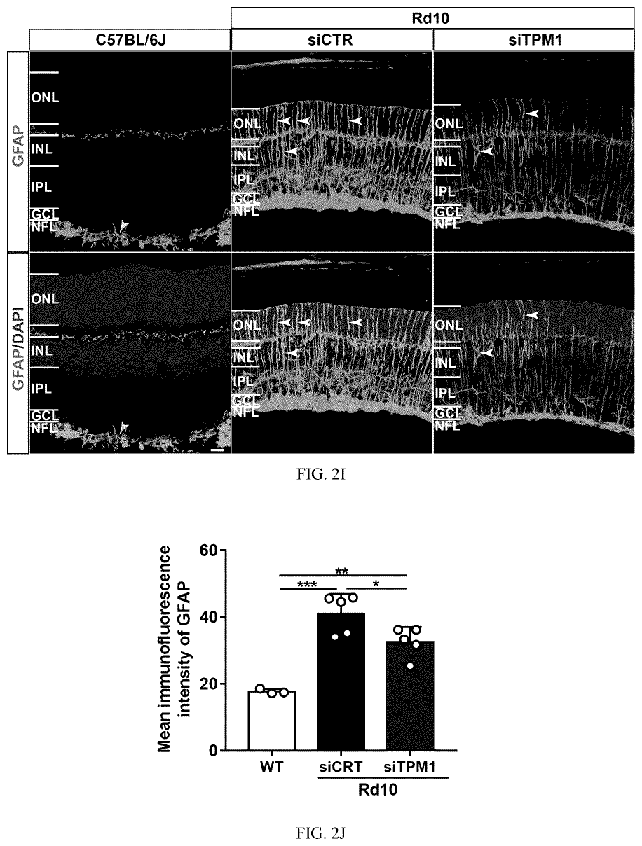

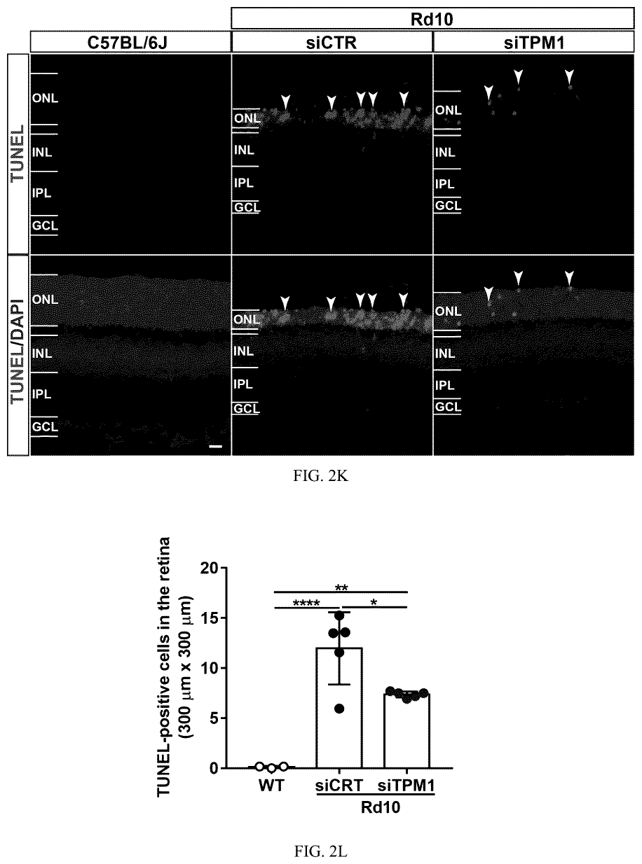

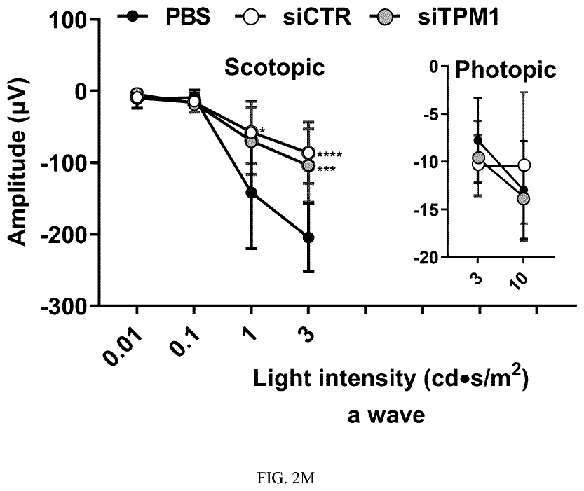

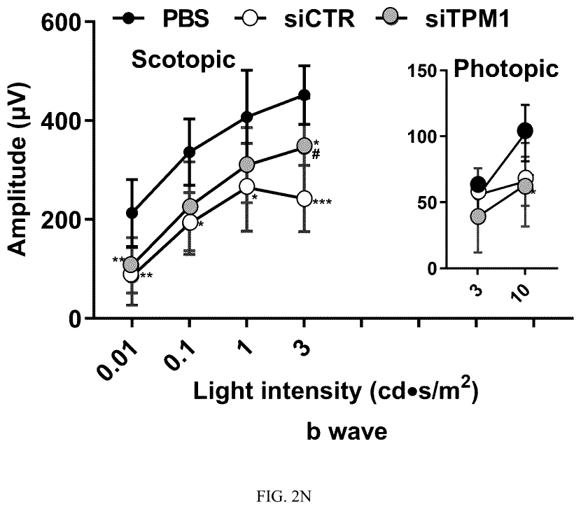

The results reveal a persistent increase in TPM1 protein levels in rd10 mice from P19, with significant elevation at P22 and P25 compared to age-matched WT mice (FIGS. 1A-1B). This upregulation was consistent with transcriptomic data (FIG. 1C and FIG. 2A). These findings suggest that TPM1 is intricately involved in the pathological progression of RP in the rd10 mouse model.

### Example 2. Genetic Knockdown of TPM1 Ameliorates Inflammation and Photoreceptor Degeneration in rd10 Mice

To explore the role of TPM1 during photoreceptor degeneration in RP, TPM1 is genetically knocked down by intravitreal injection TPM1-specific siRNA in rd10 mice. Three siRNA pools targeting TPM1 (siTPM1) and negative siRNA are synthesized by Synbio Technologies (China), siRNA sequence as follows:

siTPM1-1 (Sense):
(SEQ ID NO: 17)
GAUGAACUGGACAAAUACUTT;
siTPM1-1 (Anti-sense):
(SEQ ID NO: 18)
AGUAUUUGUCCAGUUCAUCTT;
siTPM1-2 (Sense):
(SEQ ID NO: 19)
GAUCAGUAACCAAAUUGGATT;
siTPM1-2 (Anti-sense):
(SEQ ID NO: 20)
UCCAAUUUGGUUACUGAUCTT;
siTPM1-3 (Sense):
(SEQ ID NO: 21)
GAAAAGCAUUGAUGACUUATT;
siTPM1-3 (Anti-sense):
(SEQ ID NO: 22)
UAAGUCAUCAAUGCUUUUCTT;
Negative siRNA (Sense):
(SEQ ID NO: 23)
UUCUCCGAACGUGUCACGU with 3′ modification of
terminal deoxynucleotidyl transferase recognition
site;
and
Negative siRNA (Anti-sense):
(SEQ ID NO: 24)
ACGUGACACGUUCGGAGAA with 3′ modification of
terminal deoxynucleotidyl transferase recognition
site.

In brief, 846 bp fragments of TPM1 (NM_001164255.1) from the start codon are subcloned into the vector p-YOE-PR006 (UBIGENE, China). This vector, featuring a ubiquitous CAG promotor to drive TPM1 expression and enhanced green fluorescence protein (EGFP) expression, is then used for the creation of pCAG-mTPM1-EGFP and pCAG-EGFP (negative control). Subsequently, these constructs are packaged with AAV5 serotype (UBIGENE, China), and the concentration of AAV stocks is adjusted to 1×10¹³ genome copies/ml.

For the intravitreal siRNA injection, mice are anesthetized with a mixture of ketamine hydrochloride (100 mg/kg) and xylazine (20 mg/kg) and kept under a dissecting microscope. After topical application of 0.5% Alcaine, a sterile, sharp 31 G needle is used to make an incision at the superior nasal sclera. A glass pipette is then inserted into the incision, and approximately 1 μl of siRNA solution is slowly injected into the vitreous. After injection, mice are kept on a heat pad until complete recovery. This procedure, performed three times in rd10 and TREM2-rd10 mice from P16 at two days intervals, preceded the collection of retinas at P25.

Following photoreceptor degeneration of RP, mutant proteins such as PDE6B, trigger inflammation response to further exacerbate pathological progression of RP (Ferrari et al., 2011; Liu et al., 2022; Ortega and Jastrzebska, 2021). Indeed, the present invention shows that pro-inflammatory cytokines (TNF-a, IL-1β and IL-6) and chemokines (COX-2 and iNOS) in rd10 retinas at P25 are remarkably elevated compared to age-matched WT retinas (FIGS. 2B-2F), indicating the elevation of inflammation. However, TPM1 knockdown in rd10 retinas significantly decreases the transcriptomic levels of pro-inflammatory cytokine IL-6 and chemokines COX-2 and iNOS compared to siCTR-treated group (FIGS. 2A and 2D-2F), suggesting that TPM1 might regulate inflammation in rd10 mice.

It is demonstrated that microglia activation coincided with photoreceptor apoptosis, contributing to the progression of RP (Kaur and Singh, 2021; Ortega and Jastrzebska, 2021; Peng et al., 2014; Rashid et al., 2019). To explore microglia activation, immunohistochemistry with Iba-1 (a specific marker for microglia) and CD68 (a marker for activated microglia) antibodies is performed. It is found that more microglia in rd10 retinas at P25 is positive for CD68 and presents a morphological alteration from a ramified appearance to an amocboid shape compared to age-matched WT retinas (FIGS. 2G-2H), and activated microglia migrates into the outer nuclear layer (ONL) from their normal localization within outer plexiform layer (IPL) (FIGS. 2G-2H), indicating microglia activation. Following TPM1 knockdown by treatment with TPM1 specific siRNA in rd10 mice, the number of activated microglia migrated into ONL are significantly decreased compared to siCTR-treated group at P25 (FIGS. 2G-2H), indicating that TPM1 regulates microglia activation during photoreceptor degeneration.

In healthy retina, astrocytes are entirely restricted to the nerve fiber layer (NFL). In RP, photoreceptor loss triggers astrocytes and Müller cells activation with increased GFAP immunoreactivity. The processes of reactive astrocytes becomes thicker, and many of thickened astrocytic processes extends into the inner plexiform layer (IPL) from their original tiled NFL (Fernández-Sánchez et al., 2015; Li et al., 2022a). In line with these observations, it is found that astrocytes are significantly activated, and the processes of activated astrocytes migrates into the IPL, even to the ONL in rd10 retina at P25 compared to age-matched WT retinas (FIGS. 21-2J).

However, following TPM1 knockdown in rd10 mice, it is found that activated astrocytes are inhibited with decreased GFAP immunoreactivity and less astrocytic processes extends into the IPL and ONL compared to siCTR-treated group (FIGS. 2I-2J), suggesting that TPM1 may be involved in regulating astrocytes activation in rd10 mice. Together these results indicate that elevated TPM1 in rd10 retinas contributes to neuroinflammation.

Increasing evidence reported that activated microglia could phagocytize apoptotic photoreceptors to exacerbate photoreceptor degeneration in RP (Guo et al., 2022; Hickman and Izzy, 2018; Rashid et al., 2019). As the present invention demonstrates that TPM1 regulates microglia activation during photoreceptor degeneration in rd10 retina, it is next to explore if TPM1 elevation in rd10 contributes to photoreceptor death. Therefore, the Terminal deoxynucleotidyl transferase (TdT)-mediated biotinylated deoxyuridine-triphosphate (dUTP)-biotin nick-end labeling (TUNEL) assay is performed. It is shown that TUNEL-positive cells in the ONL are significantly increased in rd10 mice at P25 compared to age-matched WT mice (FIGS. 2K-2L), indicating photoreceptor death, which is consistent with previous studies (Murakami et al., 2013; Newton and Megaw, 2020; Sanges et al., 2006). However, TPM1 knockdown following siTPM1 injection in rd 10 mice significantly decreases TUNEL-positive cells compared to siCTR-treated group (FIGS. 2K-2L), indicating that TPM1 is involved in regulating photoreceptor survival in rd10 mice.

Given the role of TPM1 in neuroinflammation and photoreceptor loss in rd10, the affection of TPM1 on visual function during photoreceptor degeneration is further evaluated using the electroretinographic (ERG) analysis. Briefly, mice are anesthetized with a mixture of ketamine hydrochloride (100 mg/kg) and xylazine (20 mg/kg) after dark adaptation overnight. And then, the pupils are dilated with 1% mydriacyl (Alcon) and the eyes are kept moist by treating with 3% Hypromellose lubricating gel solution in both corneas before performing the ERG recording using the Celeris ERG system (Diagnosys, USA). The scotopic ERG is firstly performed with different light intensities at 0.01, 0.1, 1, and 3 cd·s/m². After 10 min light adaptation under background intensity at 30 cd·s/m², photopic ERG is recorded at 3 and 10 cd·s/m² light intensities. Ten sweeps are acquired with each light stimulus. The distance between the baseline and the negative peak are measured as the amplitude of ERG a-wave, and the amplitude of b-wave is calculated between the bottom of the a-wave and the top of the tallest curve.

It is shown that amplitudes of a-wave and b-wave were constantly declined with light intensity in rd10 mice at P25 compared to age-matched WT mice (FIGS. 2M-2N), indicating the functional decline in rd10 mice. Interestingly, when TPM1 specific siRNA is intravitreally injected into rd10 mice, TPM1 knockdown in rd10 mice counteracts the decrease of amplitudes of a- and b-waves, especially in scotopic condition at 3 cd·s/m² light intensity (FIGS. 2M-2N), indicating that elevated TPM1 contributes to visual loss in rd10 mice.

Taken together these results demonstrate that elevated TPM1 during photoreceptor degeneration is responsible for inflammation, photoreceptor death and vision decline in RP.

### Example 3. TPM1 overexpression Exacerbates the Retinal Glial Reaction and Photoreceptor Loss in rd10 Mice

To further explore the role of TPM1 in regulating inflammation and photoreceptor survival in the progression of RP, CAG-mTPM1 -EGFP plasmid driven by the ubiquitous CAG promotor to overexpress TPM1 is constructed, and then pCAG-mTPM1-EGFP and pCAG-EGFP (negative control) are packaged with AAV5 serotype (UBIGENE). AAV-EGFP-TPM1 is delivered to rd10 mice through subretinal injection. Briefly, the pups at P1 or P2 are anesthetized by putting them on ice for several minutes. And then, an approximate 1.5 mm incision along the closed lid fissure are made with a straight Vannas scissors or a surgical blade under a dissecting microscope, followed by pulling apart the eyelids with a curved forceps to fully expose the eyes for injection. Around 0.4 μl AAV-EGFP-TPM1 or AAV-EGFP is injected into the subretinal space using a pulled angled glass pipette controlled by a FemtoJet® 4i (Eppendorf). After injection, gently pushing eye back behind the lids and into the orbit with curved forceps and treating some erythromycin eye ointment in the incision of lids to avoid infection. And then, the pups are kept on a heat pad at 37° C. until complete recovery.

As used herein, the term “subretinal space” refers to a space located between the neural retina and the retinal pigment epithelium (RPE).

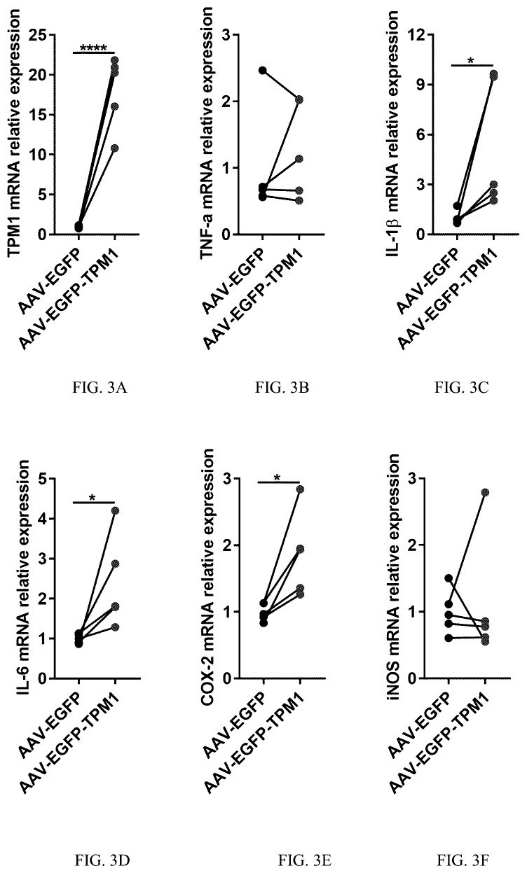

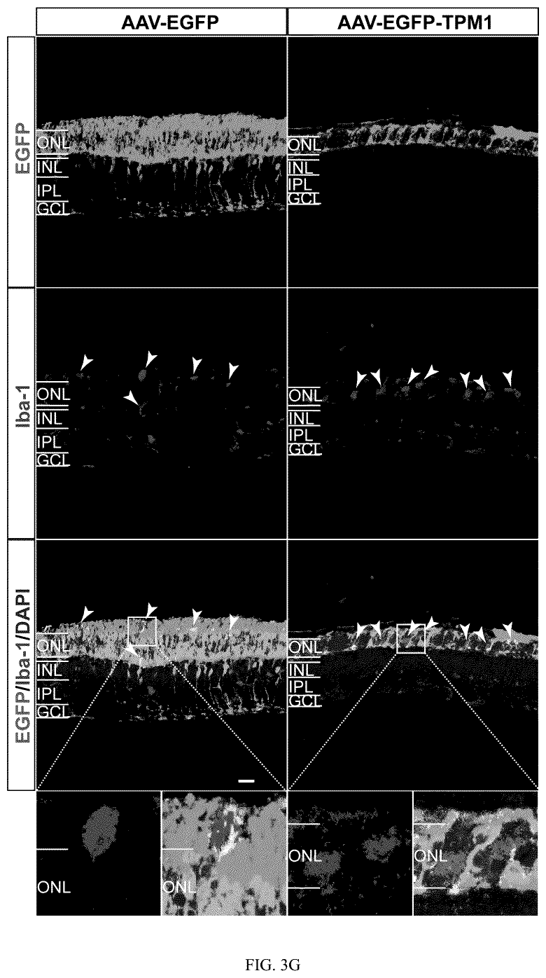

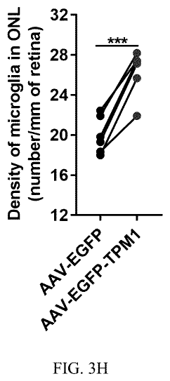

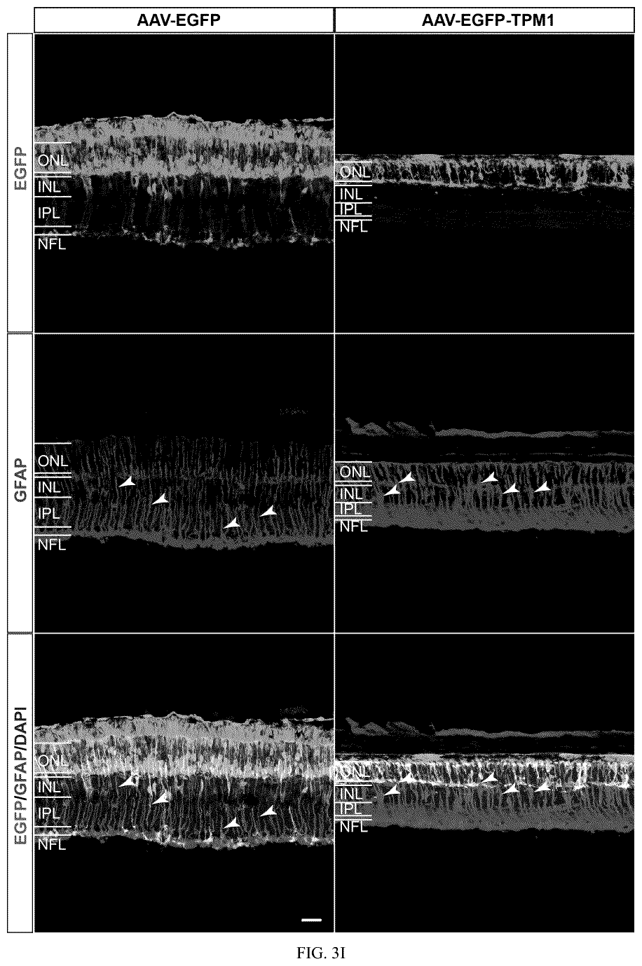

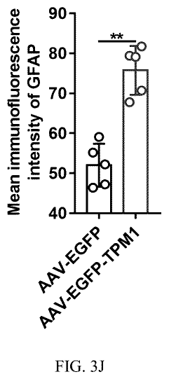

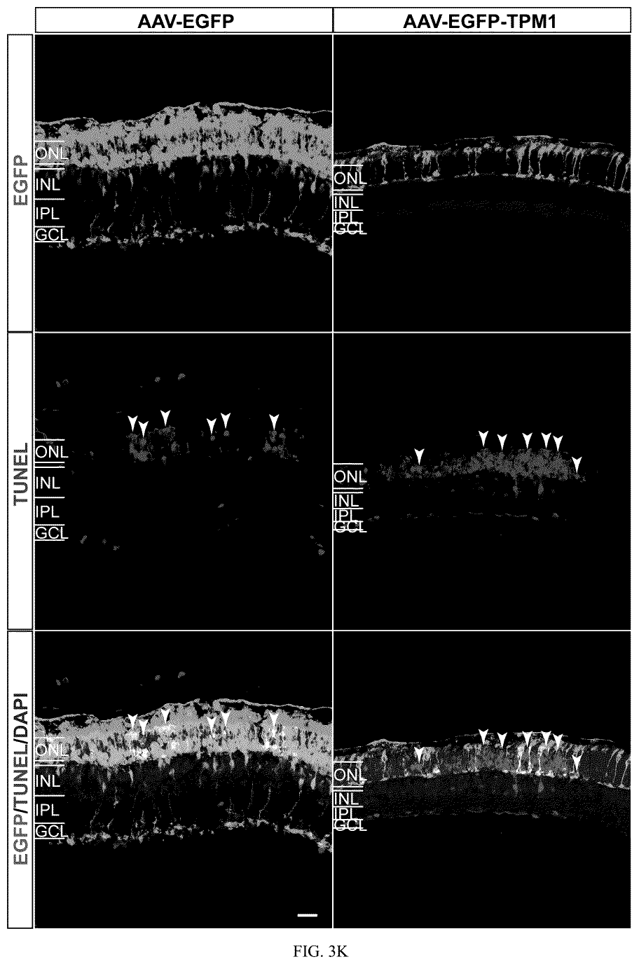

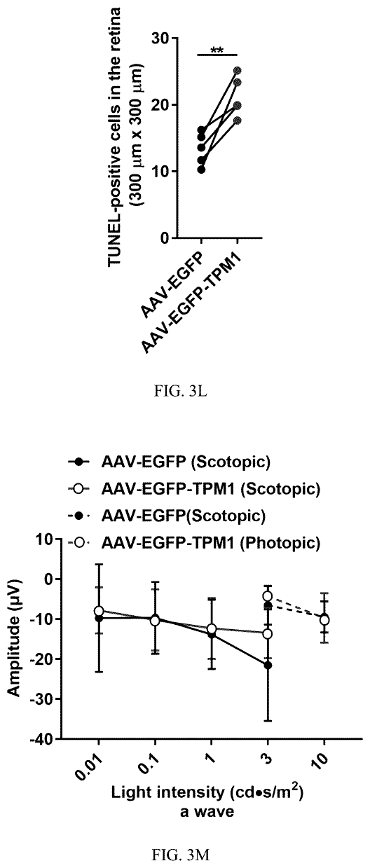

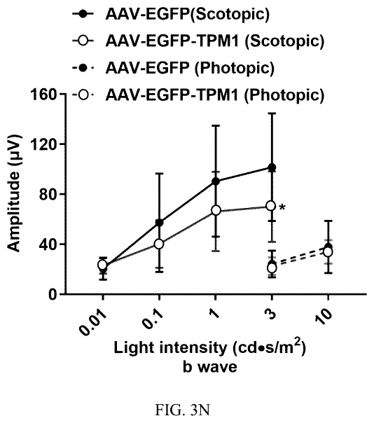

By subretinal injection AAV-EGFP-TPM1 in rd10 mice, TPMv overexpression significantly increases the transcriptomic levels of pro-inflammatory cytokines IL-1β and IL-6 and chemokine COX-2 compared to AAV-EGFP-treated group (FIGS. 3A-3F), indicating that TPM1 upregulation may deteriorate inflammation. In addition, TPM1 overexpression in rd10 retinas significantly enhances the migration of activated microglia, and more activated microglia migrates into the ONL from their normal strata within the OPL (FIGS. 3G-3H). Meanwhile, it is observed that TPM1 elevation strengthens astrocytes activation, and activated astrocytes present more thicker processes and hypertrophic morphology, and more activated astrocytic processes extends into the ONL compared to the control group (FIGS. 3I-3J). Together these results indicate TPM1 increase in rd10 retinas may reinforce the retinal glial reaction.

In addition, terminal deoxynucleotidyl transferase (TdT)-mediated biotinylated deoxyuridine-triphosphate (dUTP)-biotin nick-end labeling (TUNEL) assay is conducted to detect the apoptotic cells. In brief, the sections are fixed with 4% PFA at 37° C. for 15 min followed by permeabilization with proteinase K for 15 min at room temperature. And then, the sections are incubated with TdT reaction mixture for 1 h at 37° C. After rinsing with PBS for three times, the sections are incubated with TUNEL reaction cocktails containing Alexa Fluor™ 594 Picolyl Azide and 10X Click-iT™ Plus TUNEL Reaction buffer additive (Invitrogen) for 30 min at 37° C. Following several washes with 3% BSA in PBS, the slides are incubated with DAPI (Sigma) for 15-20 min and mounted with Dako fluorescence mounting medium. For quantification of TUNEL-positive cells, three views in each retinal section at 100 μm (central), 1 mm (middle) and 1.8 mm (peripheral) from the optical nerve head are captured, and TUNEL-positive cells are manually counted.

The TUNEL experiments reveal that TPM1 overexpression in rd10 mice by subretinal injection with AAVE-EGFP-TPM1 increases the number of TUNEL-positive cells compared to AAV-EGFP-treated group (FIGS. 3K-3L), suggesting that TPM1 elevation contributes to the deterioration of photoreceptor death. ERG recording shows markedly reduced amplitudes of scotopic b-wave, especially at 3 cd·s/m² light intensity in AAV-EGFP-TPM1-treated retinas compared to AAV-EGFP treated retinas in rd10 mice (FIGS. 3M-3N), indicating that increased TPM1 in rd10 retinas is associated with vision decline.

Collectively, these results further verify that TPM1 might directly mediate neuroinflammation and photoreceptor apoptosis during photoreceptor degeneration in RP.

### Example 4. TREM2 Knockout Downregulates TPM1 and Inhibits the Retinal Glial Reaction snd Photoreceptor Degeneration in rd10 Mice

Triggering receptor expressed on myeloid cells 2 (TREM2), exclusively expressed by microglia, plays an important role in regulating neuroinflammation in the CNS (Li and Zhang, 2018; Walter, 2016), TPM1 mediates inflammation downstream of TREM2 via the PKA/CREB signaling pathway (Li et al., 2022b).

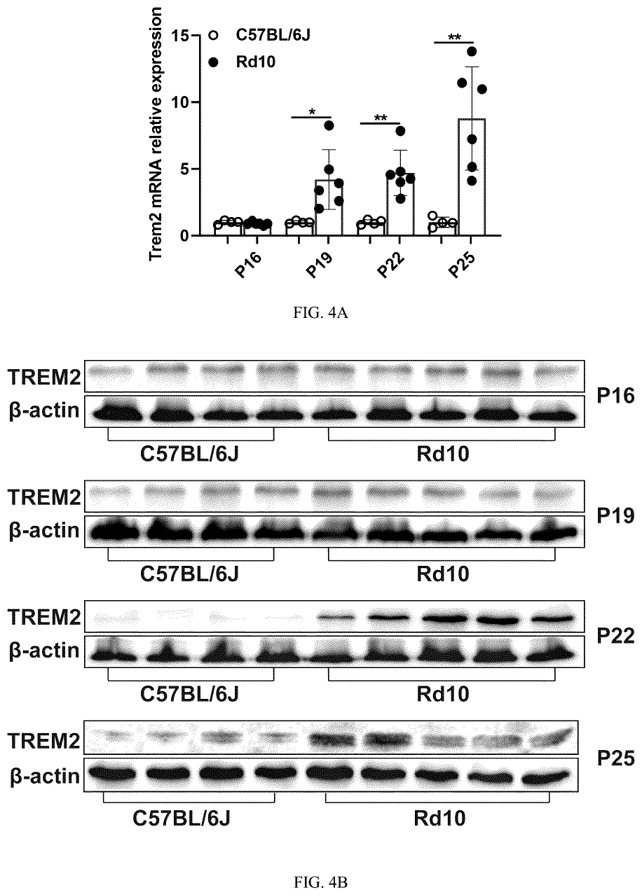

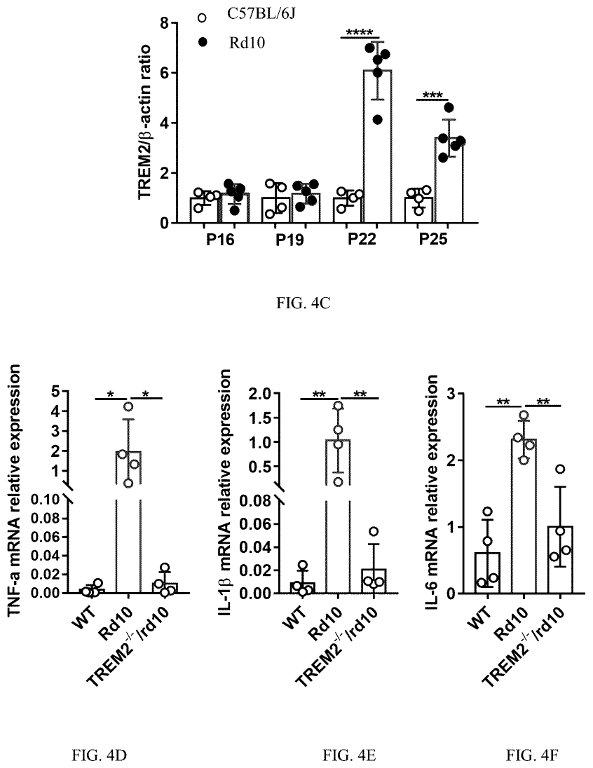

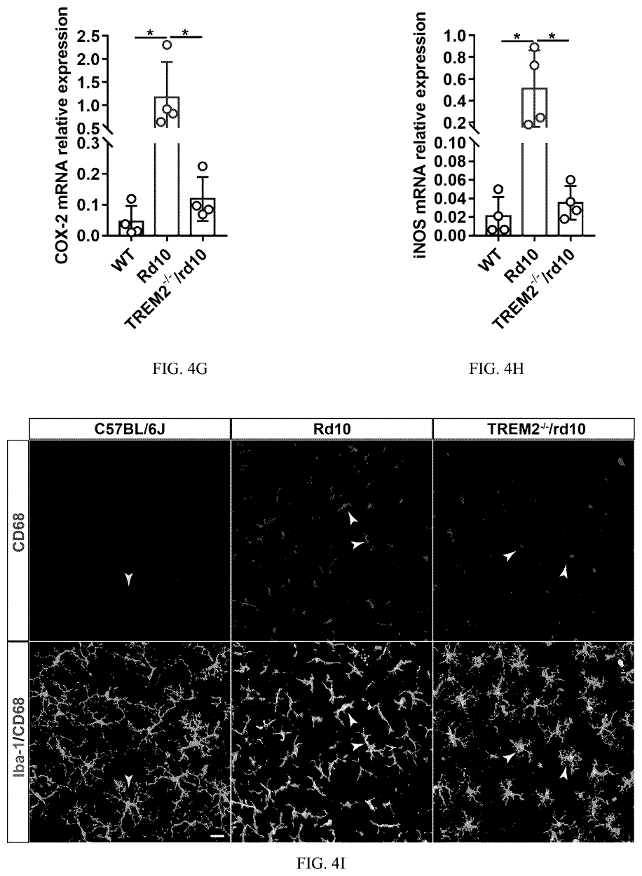

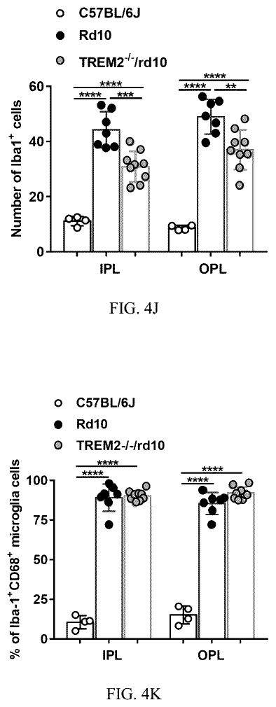

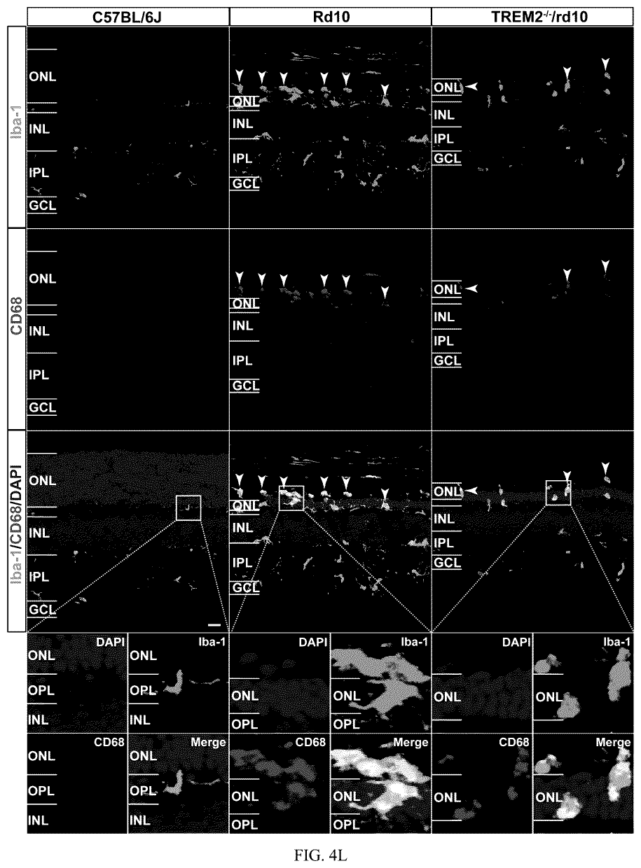

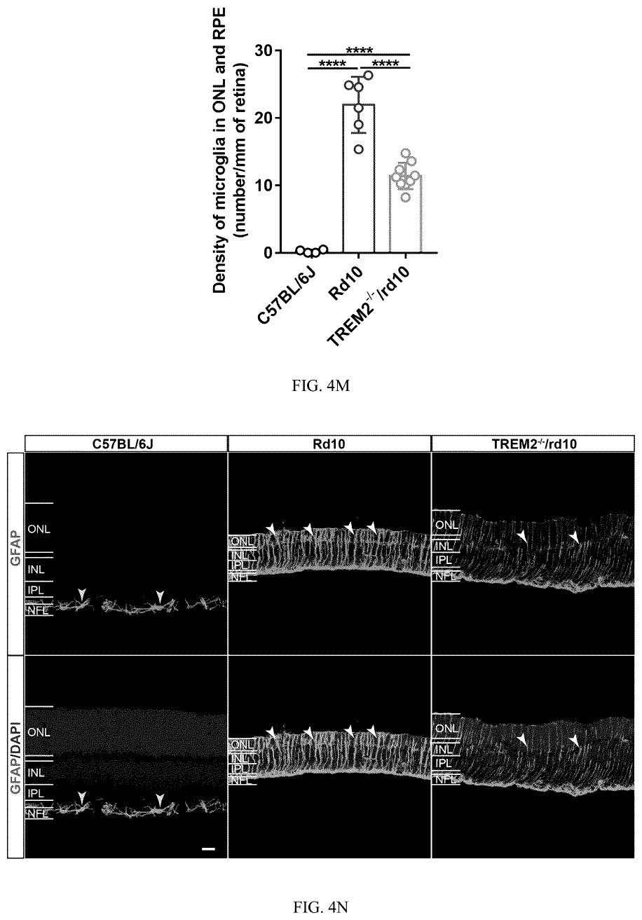

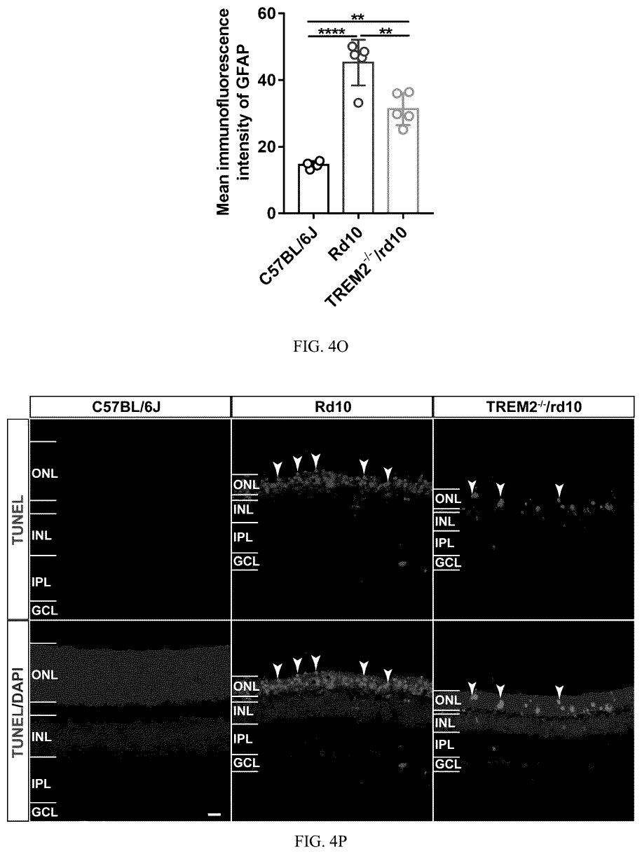

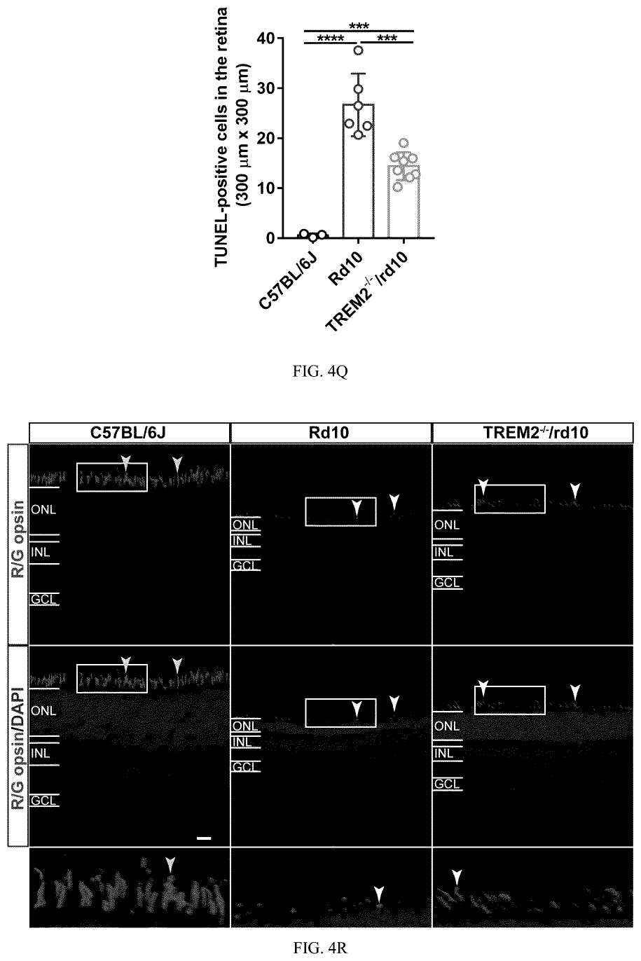

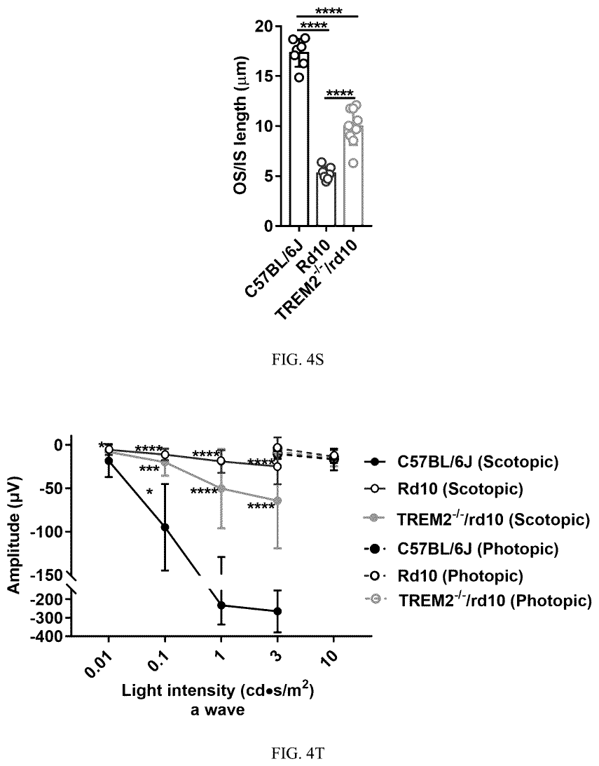

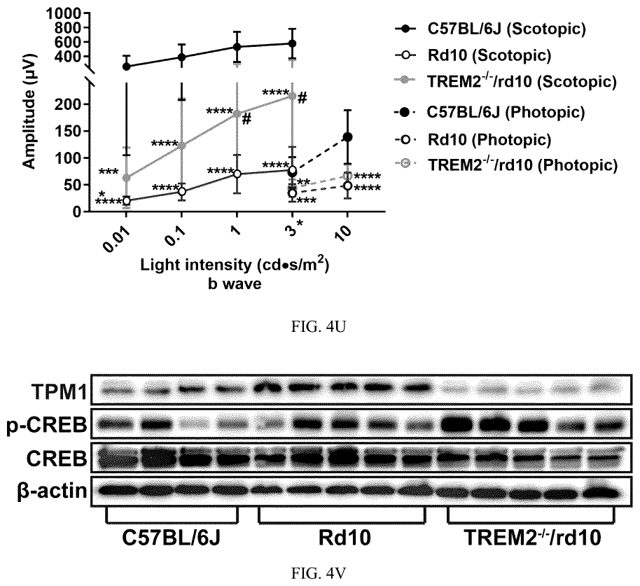

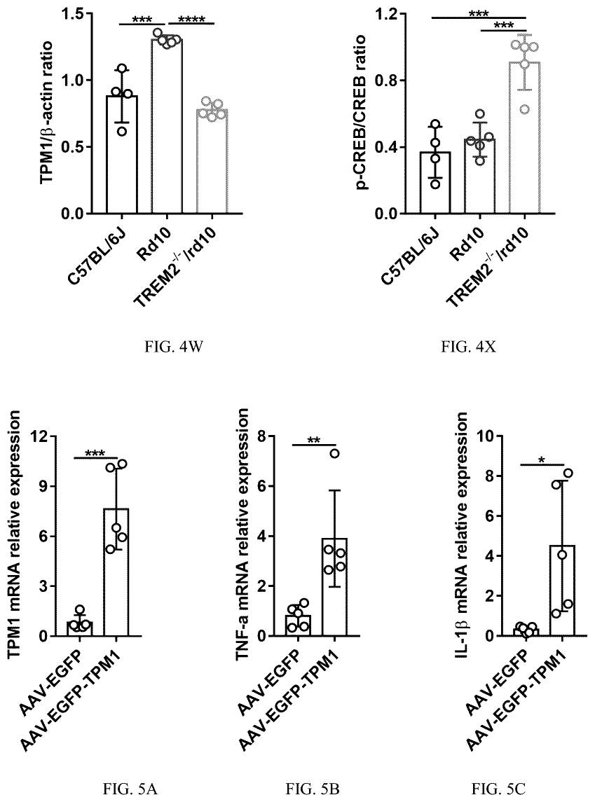

The role of TREM2 in TPM1-regulated inflammation and photoreceptor death in RP is investigated in the present invention. The temporal expression pattern of TREM2 is detected during photoreceptor degeneration in a mouse model of RP. The results show that the transcriptomic level of TREM2 is continuously increased with photoreceptor death in rd10 (FIG. 4A), and the protein level of TREM2 is also significantly elevated in rd10 retinas at P22 and P25 compared to age-matched WT retinas (FIGS. 4B-4C), suggesting that elevated TREM2 may be involved in the pathological process of RP.

To further explore the role of TREM2 in RP, TREM2^{-/-} mice (Jackson Laboratory, Stock no: 027197) are backcrossed with rd10 mice to generate TREM2^{-/-}/rd10 mice. TREM2 knockout in rd10 mice significantly reduces the transcriptomic levels of pro-inflammatory cytokines TNF-a, IL-1β and IL-6 and chemokines COX-2 and iNOS compared to age-matched rd10 mice at P25 (FIGS. 4D-4H), indicating that TREM2 deficiency may counteract inflammation which is induced by photoreceptor degeneration in RP. Furthermore, TREM2 knockout in rd10 mice at P25 significantly decreases the number of Iba1+ microglia in both IPL and OPL (FIGS. 4I-4K), and inhibits the migration of activated microglia into the ONL (FIGS. 4L-4M).

Similarly, it is observed that TREM2 knockout in rd10 mice markedly deactivates astrocytes with decreased GFAP immunoreactivity and thinner processes compared to age-matched rd10 mice at P25 (FIGS. 4N-4O). Together these results indicate that TREM2 deficiency in rd10 mice suppresses the retinal glial reaction. As a result of inflammation inhibition, TREM2 knockout in rd 10 mice decreases the number of TUNEL-positive cells (FIGS. 4P-4Q) and partially rescues cone photoreceptor loss (FIGS. 4R-4S) compared to age-matched rd10 mice at P25, indicating that TREM2 deficiency might be beneficial for photoreceptor survival in RP. Functionally, it is found that TREM2 knockout in rd10 mice significantly increases the amplitudes of b-wave under scotopic condition with different light intensities, especially at 1 and 3 cd·s/m² light intensities compared to age-matched rd10 mice at P25 (FIGS. 4T-4U), indicating that TREM2 deficiency in rd10 mice could slow down vision decline.

It is known that the activation of CREB signal plays a role in inhibiting inflammation, the TPM1 elevation contributes to inflammation, and TPM1 regulates CREB signal in mediating inflammation. Also, TPM1 mediates inflammation downstream of TREM2 via PKA/CREB signaling pathway. Interestingly, in the present invention, TREM2 knockout in rd10 mice remarkably decreases the protein level of TPM1 and increases the expression of phosphorylated CREB compared to age-matched rd10 mice at P25 (FIGS. 4V-4X), indicating that TREM2 may regulate TPM1/CREB signal during photoreceptor degeneration in RP. Together these findings indicated that TREM2 deficiency in rd10 mice may partially rescue photoreceptor degeneration by regulating TPM1 /CREB signaling pathway.

### Example 5. Genetic Ooverexpression of TPM1 in TREM2^{-/-}/rd10 Mice Increases Inflammation and Photoreceptor Loss

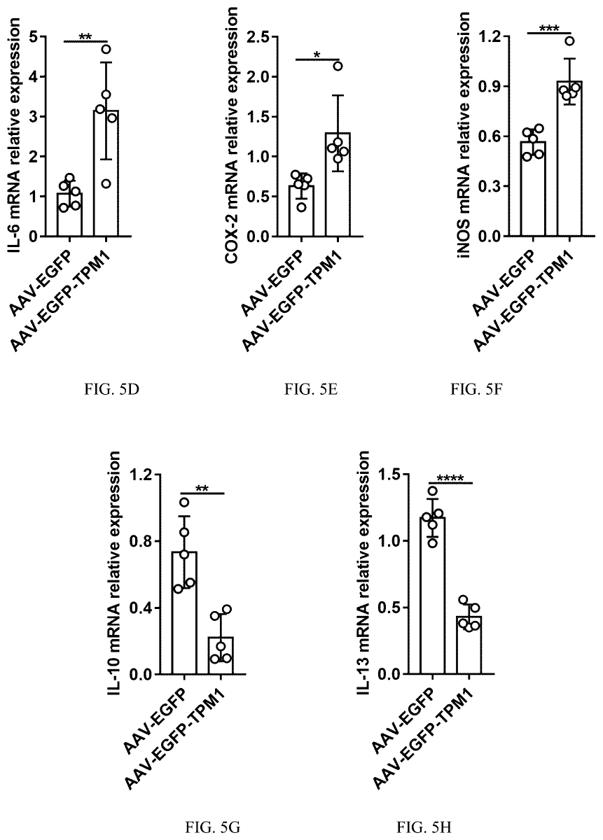

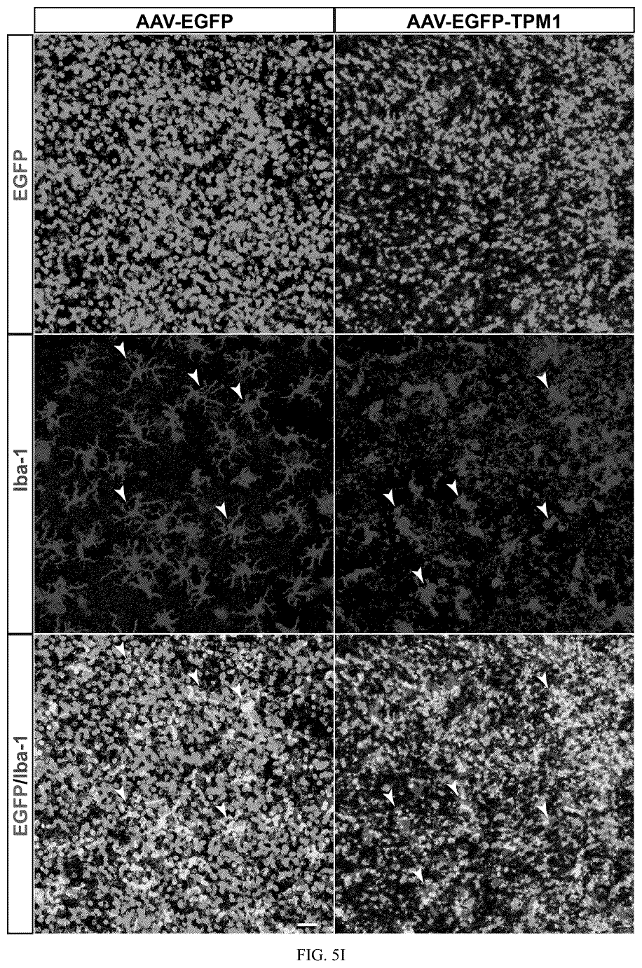

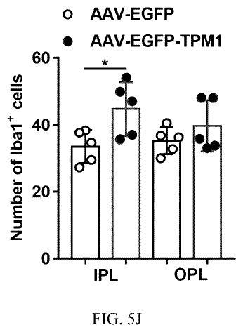

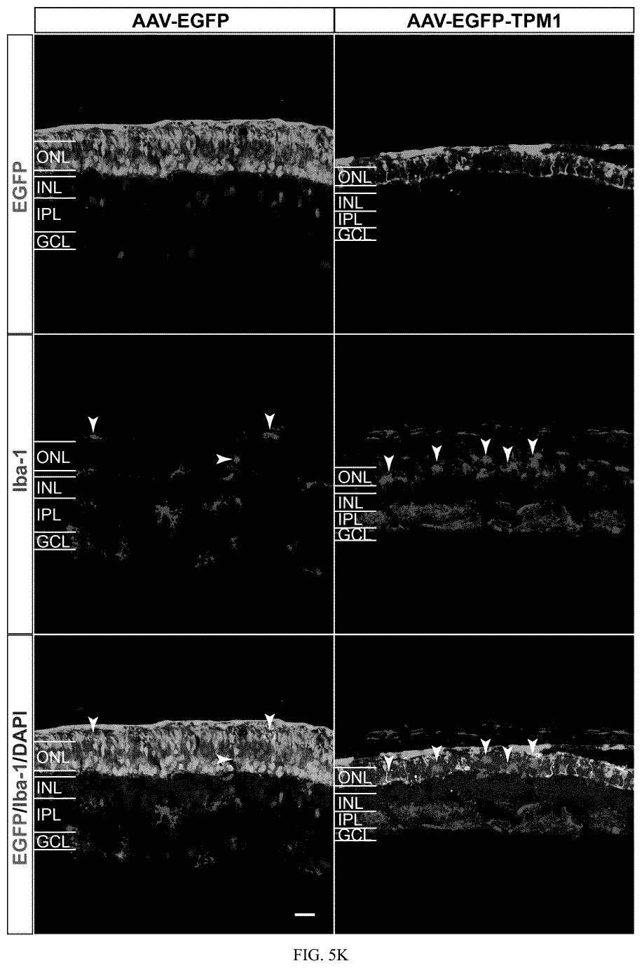

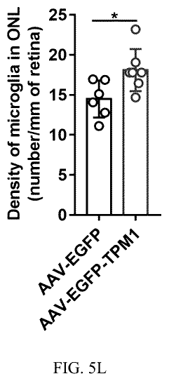

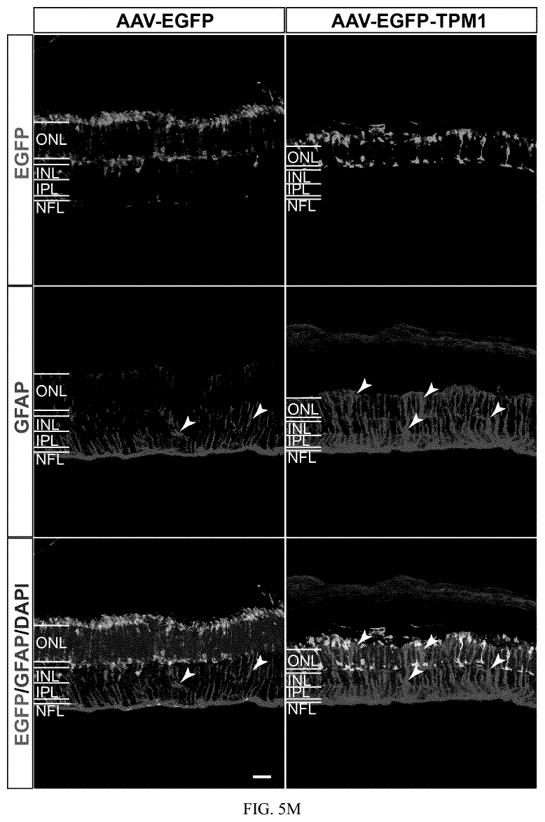

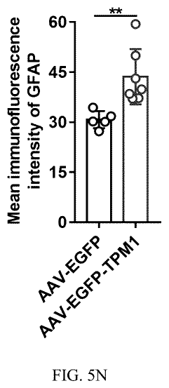

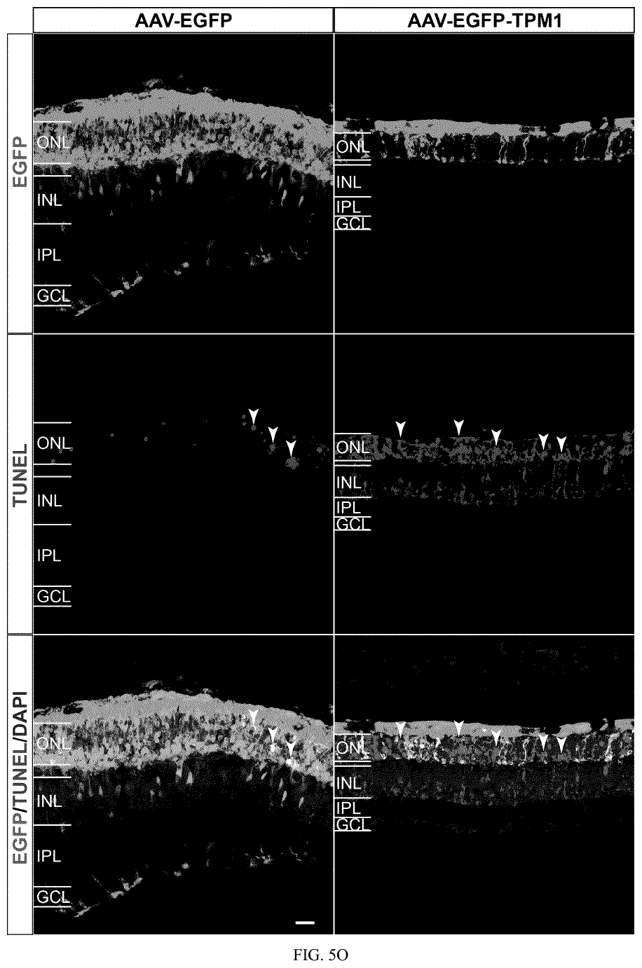

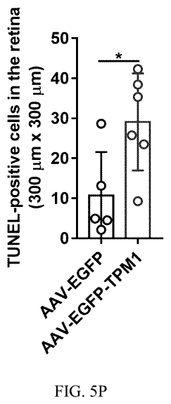

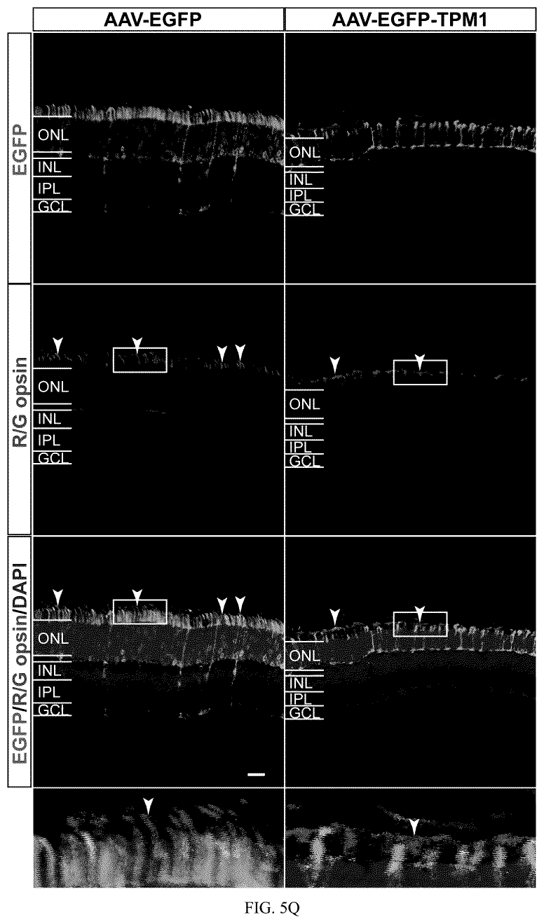

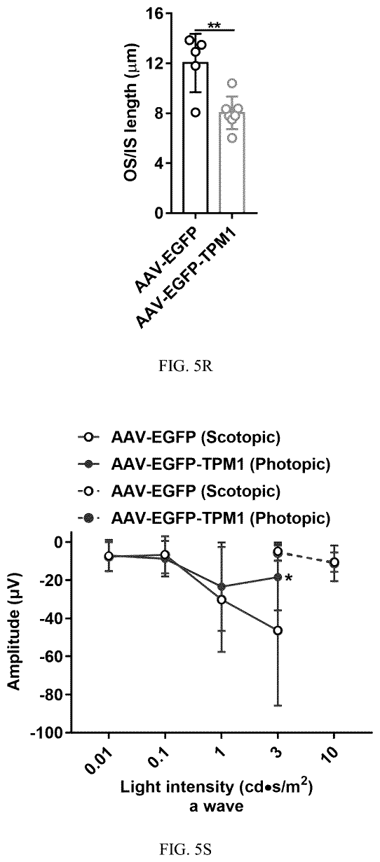

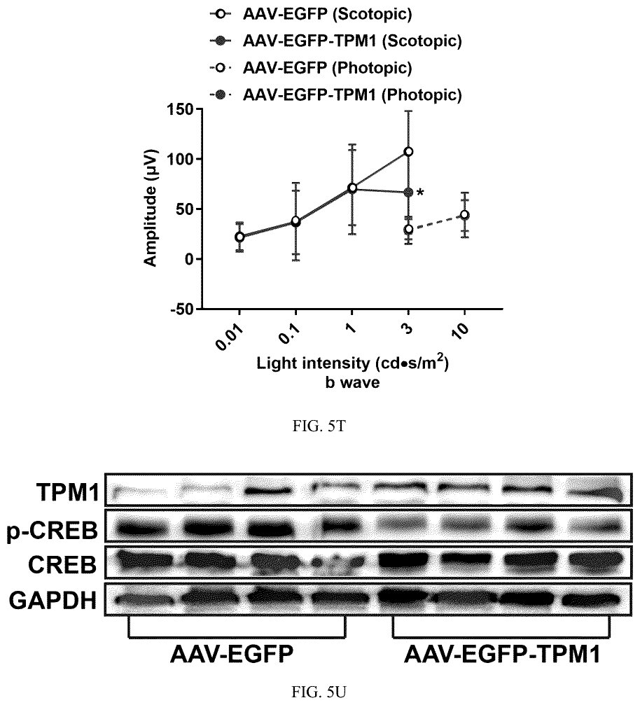

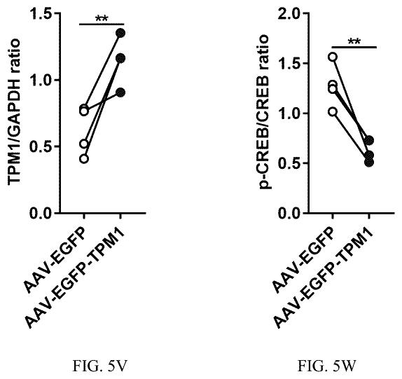

To further study the role of TPM1 in TREM2-mediated inflammation in RP, TPM1 by subretinal injection of AAV-EGFP-TPM1 is overexpressed in TREM2^{-/-}/rd10 mice. The results show that TPM1 overexpression in TREM2^{-/-}/rd10 mice at P25 significantly increases the transcriptomic levels of pro-inflammatory cytokines TNF-a, IL-1β and IL-6 and chemokines COX-2 and iNOS, and decreases anti-inflammatory cytokines IL-10 and IL-13 compared to AAV-EGFP treated control group (FIGS. 5A-5H), indicating that TPM1 elevation aggravates inflammation in TREM2-deficient rd10 retinas. Furthermore, TPM1 overexpression in TREM2^{-/-}/rd10 mice increases the number of Iba1+ microglia in the IPL (FIGS. 5I-5J), and more activated microglia migrated into the ONL (FIGS. 5K-5L). Meanwhile, the TPM1 elevation in TREM2^{-/-}/rd10 mice at P25 significantly enhances astrocytes activation with increased GFAP immunoreactivity and thicker processes, and more astrocytic processes extends into the IPL and ONL compared to AAV-EGFP-treated control group (FIGS. 5M-5N). These results indicate that TPM1 increase in TREM2-deficient rd10 mice intensifies the retinal glial reaction.

Moreover, it is observed that TPM1 overexpression in TREM2^{-/-}/rd10 retinas by subretinal injection with AAV-EGFP-TPM1 significantly increases the number of TUNEL-positive cells (FIGS. 5O-5P) and decreases the number of cone photoreceptor (FIGS. 5Q-5R) compared to AAV-EGFP-treated control retinas, indicating that TPM1 elevation in TREM2-deficient rd10 retinas strengthens photoreceptor loss. ERG recording reveals that TPM1 increase in TREM2^{-/-}/rd10 mice obviously reduces the amplitudes of a- and b-wave under scotopic condition at 3 cd·s/m² light intensity compared to AAV-EGFP-treated control mice at P25 (FIGS. 5S-5T), suggesting that TPM1 elevation in TREM2-deficient rd10 mice contributes to functional decline.

In addition, the TPM1 overexpression in TREM2^{-/-}/rd10 retinas significantly inhibits the protein level of phosphorylated CREB (FIGS. 5U-5W), indicating that TPM1 elevation in TREM2-deficient rd10 retinas may deactivate CREB signal. Together these findings further confirm that TREM2 regulates TPM1/CREB signaling pathway in mediating inflammation and photoreceptor loss in RP.

To further explore the downstream signals of TPM1 in regulating inflammation and photoreceptor death, label-free LC-MS/MS is conducted. The proteomic analysis shows that vasorin (Vasn) is a potential molecule that is involved in TPM1-mediated neuroinflammation and photoreceptor death in RP. It will be further investigated whether Vasn knockdown or overexpression affects the role of TPM1 in RP.

Microglia-derived neuroinflammation contributes to many neurodegenerative diseases including Alzheimer diseases, Parkinson disease and dementia. TPM1 is an important regulator of pro-inflammatory genes in microglia, which can trigger changes in gene signatures in microglia, resulting in a gradual transition from homeostatic microglia to diseases-associated microglia and neuronal death in wild-type and RP retinas. Therefore, targeting TPM1 could potentially be used against various inflammation-related neurodegenerative diseases in the brain.

In summary, it is demonstrated in the present invention that TPM1 is significantly elevated in rd10 mice, a mouse model of RP. Gain-or-loss of function of TPM1 in rd10 mice shows that TPM1 directly regulates neuroinflammation and photoreceptor apoptosis during photoreceptor degeneration.

Moreover, triggering receptor expressed on myeloid cells 2 (TREM2), which is specifically expressed by microglia in the brain, is involved in regulating inflammation in rd10 mice. TREM2 knockout in rd10 mice inhibits the retinal glial reaction, inflammation and vision decline, and TPM1 overexpression enhances neuroinflammation and photoreceptor cell death and visual function loss in the rd10 retina with TREM2-deficiency, indicating that TPM1 is a potential target for combating RD progression.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of ±10%, ±5%, ±1%, or ±0.5% of the given value or range. The range may be indicated herein as from one endpoint to another endpoint or between two endpoints. Unless otherwise specified, all the ranges disclosed in the present disclosure include endpoints. When reference is made to “substantially” the same numerical value or characteristic, the term may refer to a value within ±10%, ±5%, ±1%, or ±0.5% of the average of the values.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A pharmaceutical composition comprising a tropomyosin 1 (TPM1) inhibitor for treating a retinal degeneration in a subject in need thereof, further comprising a pharmaceutically acceptable addition.

2. The pharmaceutical composition of claim 1, wherein the pharmaceutical composition suppresses and/or knocks down the expression and/or activity of the TPM1.

3. The pharmaceutical composition of claim 2, wherein the TPM1 inhibitor comprises a triggering receptor expressed on myeloid cells 2 (TREM2) silencer.

4. The pharmaceutical composition of claim 1, wherein the refractive disorder is selected from age-related macular degeneration (AMD) or retinitis pigmentosa (RP).

5. The pharmaceutical composition of claim 1, wherein the pharmaceutically acceptable addition comprises an excipient, a stability additive, a carrier, a diluent, and a solubilizer.

6. The pharmaceutical composition of claim 1, wherein the pharmaceutical composition is formulated to an administration form that enables delivery to the subject's subretinal space through the cornea and/or the blood-retinal barrier.

7. The pharmaceutical composition of claim 6, wherein the administration form is selected from an immediate-release form or a controlled-release form.

8. The pharmaceutical composition of claim 6, wherein the pharmaceutical composition is delivered through an approach selected from an intravitreal injection, a subretinal injection or a suprachoroidal injection.

9. The pharmaceutical composition of claim 6, the administration form comprises an injection form, an eye drop form, an eye ointment form, a hydrogel form, an ultrasonic ocular drug delivery form, a drug-loaded contact lenses form, a drug-eluting implant form, a nanoparticle-mediated delivery, an intravitreal gene therapy form, and an intravitreal microneedle form.

10. A method for reducing neuroinflammation in a subject in need thereof, comprising administering the composition of claim 1 to the subject.

11. A method for rescuing visual function in a subject in need thereof, comprising administering the composition of claim 1 to the subject.

12. The method of claim 11, wherein the photoreceptor degeneration is reversed in the subject.

13. Use of the pharmaceutical composition of claim 1 for treating a retinal degeneration in a subject in need thereof.

14. The use of claim 13, wherein the retinal degeneration is selected from AMD or RP.

15. The use of claim 13, wherein the pharmaceutical composition reduces neuroinflammation in the subject.

16. The use of claim 13, wherein the pharmaceutical composition reverses the photoreceptor degeneration and rescues visual function in the subject.


SEQUENCE LISTING (24 sequences)

SEQ ID NO: 1 - DNA, 21 bp, synthetic construct
  source 1..21: mol_type = other DNA
ctgaagatgctgaccggaagt

SEQ ID NO: 2 - DNA, 25 bp, synthetic construct
  source 1..25: mol_type = other DNA
tggttactgatctctctgcaaactc

SEQ ID NO: 3 - DNA, 20 bp, synthetic construct
  source 1..20: mol_type = other DNA
atggcctccctctcatcagt

SEQ ID NO: 4 - DNA, 20 bp, synthetic construct
  source 1..20: mol_type = other DNA
tttgctacgacgtgggctac

SEQ ID NO: 5 - DNA, 23 bp, synthetic construct
  source 1..23: mol_type = other DNA
aatgccaccttttgacagtgatg

SEQ ID NO: 6 - DNA, 20 bp, synthetic construct
  source 1..20: mol_type = other DNA
ggaaggtccacgggaaagac

SEQ ID NO: 7 - DNA, 20 bp, synthetic construct
  source 1..20: mol_type = other DNA
gggactgatgctggtgacaa

SEQ ID NO: 8 - DNA, 22 bp, synthetic construct
  source 1..22: mol_type = other DNA
agcattggaaattggggtagga

SEQ ID NO: 9 - DNA, 20 bp, synthetic construct
  source 1..20: mol_type = other DNA
cttcgggagcacaacagagt

SEQ ID NO: 10 - DNA, 20 bp, synthetic construct
  source 1..20: mol_type = other DNA
aagtggtaaccgctcaggtg

SEQ ID NO: 11 - DNA, 20 bp, synthetic construct
  source 1..20: mol_type = other DNA
cttgctggaaccgtcaccat

SEQ ID NO: 12 - DNA, 21 bp, synthetic construct
  source 1..21: mol_type = other DNA
cttgattcctggaggtgctgt

SEQ ID NO: 13 - DNA, 20 bp, synthetic construct
  source 1..20: mol_type = other DNA
ggtgagaagctgaagaccct

SEQ ID NO: 14 - DNA, 23 bp, synthetic construct
  source 1..23: mol_type = other DNA
tcattcatggccttgtagacacc

SEQ ID NO: 15 - DNA, 20 bp, synthetic construct
  source 1..20: mol_type = other DNA
cctggattccctgaccaaca

SEQ ID NO: 16 - DNA, 20 bp, synthetic construct
  source 1..20: mol_type = other DNA
attttggtatcggggaggct

SEQ ID NO: 17 - RNA, 21 nt, synthetic construct
  source 1..21: mol_type = other RNA
gatgaactggacaaatacttt

SEQ ID NO: 18 - RNA, 21 nt, synthetic construct
  source 1..21: mol_type = other RNA
agtatttgtccagttcatctt

SEQ ID NO: 19 - RNA, 21 nt, synthetic construct
  source 1..21: mol_type = other RNA
gatcagtaaccaaattggatt

SEQ ID NO: 20 - RNA, 21 nt, synthetic construct
  source 1..21: mol_type = other RNA
tccaatttggttactgatctt

SEQ ID NO: 21 - RNA, 21 nt, synthetic construct
  source 1..21: mol_type = other RNA
gaaaagcattgatgacttatt

SEQ ID NO: 22 - RNA, 21 nt, synthetic construct
  source 1..21: mol_type = other RNA
taagtcatcaatgcttttctt

SEQ ID NO: 23 - RNA, 19 nt, synthetic construct
  source 1..19: mol_type = other RNA
ttctccgaacgtgtcacgt

SEQ ID NO: 24 - RNA, 19 nt, synthetic construct
  source 1..19: mol_type = other RNA
acgtgacacgttcggagaa